(19) United States
(12) Patent Application Publication
(10) Pub. No.: US 2018/0252736 A1
(43) Pub. Date: Sep. 6, 2018

(54) CB2 RECEPTOR INTERNALIZATION

(71) Applicant: Arena Pharmaceuticals, Inc., San Diego, CA (US)

(72) Inventors: David J. Unett, San Diego, CA (US); Ibragim Gaidarov, San Diego, CA (US)

(21) Appl. No.: 15/754,605

(22) PCT Filed: Sep. 1, 2015
(86) PCT No.: PCT/US2015/047940

§ 371 Date: Feb. 23, 2018

(51) Int. Cl. G01N 33/94; A61K 31/352; A61K 31/05; A01K 67/00

(52) U.S. Cl. CPC G01N 33/948; A61K 31/352; A01K 2267/0356; A01K 67/00; G01N 2333/726; A61K 31/05

(57) ABSTRACT

Certain compounds and pharmaceutical compositions thereof that modulate the activity of the cannabinoid CB2 receptor, including its internalization in a cell, are provided. Certain methods of identifying compounds that increase CB2 receptor internalization are also provided. Certain compounds described herein and pharmaceutical compositions thereof are useful for the treatment of CB2 receptor-mediated disorders, such as: pain, osteoarthritis, liver fibrosis, primary biliary cirrhosis, nonalcoholic steatohepatitis, diabetic neuropathy, endometriosis, and interstitial cystitis.

Cannabinoids are a group of extracellular signaling molecules that are found in both plants and animals. Signals from these molecules are mediated in animals by two G-protein coupled receptors, Cannabinoid Receptor 1 (CB1) and Cannabinoid Receptor 2 (CB2). CB1 is expressed most abundantly in the neurons of the central nervous system (CNS), but is also present at lower concentrations in a variety of peripheral tissues and cells (Nature 346:561-564, 1990). In contrast, CB2 is expressed predominantly, although not exclusively, in non-neural tissues, e.g. in hematopoietic cells, endothelial cells, osteoblasts, osteoclasts, the endocrine pancreas, and cancerous cell lines (Nature 365:61-65, 1993; and as reviewed in Pharmacol. Rev. 58(3): 389-462, 2006). As such, CB1 is believed to be primarily responsible for mediating the psychotropic effects of cannabinoids on the body, whereas CB2 is believed to be primarily responsible for most of their non-neural effects.

There is a need for methods to identify compounds that modulate the activity of the CB2 receptor, including CB2 receptor agonists for use in safe and effective therapies. The methods described herein satisfy this need and provide related advantages as well.

### SUMMARY

Despite attempts to develop CB2 receptor agonists for various therapies, these agonists have largely failed to advance through clinical development. In some instances, CB2 receptor agonists have failed to meet a primary endpoint in clinical trials. In some instances, CB2 receptor agonists have demonstrated unwanted psychotropic effects. Prior to the present disclosure, it was unclear how to select CB2 receptor agonists to achieve sustained in vivo efficacy and avoid unwanted side effects.

Described herein is the discovery that CB2 receptors undergo rapid desensitization that is correlated with tachyphylaxis. For example, in rodent pain models, the analgesic effects of several CB2 receptor agonists disappeared only one hour after dosing. Pharmacokinetic studies demonstrated that this loss of in vivo efficacy was not due to elimination of the agonist. In fact, in some instances, the analgesic efficacy of the agonist was lost before plasma concentration of the agonist began to decline, or even while plasma concentrations were still increasing.

Further described herein is the discovery that CB2 receptor agonists must induce robust internalization of the receptor in order to maintain a sufficient level of signaling for sustained in vivo efficacy. Agonists inducing less than full CB2 receptor internalization were found to rapidly lose in vivo efficacy, while agonists inducing full CB2 receptor internalization were found to exhibit sustained in vivo efficacy. In addition, conventional receptor activation assays were found to routinely overestimate CB2 receptor agonist effects. For example, compounds exhibiting full efficacy in cAMP assays failed to drive robust receptor internalization. Although β-arrestin recruitment assays provided better predictive value for receptor internalization, some CB2 receptor agonists with robust β-arrestin efficacy failed to produce robust receptor internalization. Although full activation of the CB2 receptor appears to be necessary for receptor internalization (and therefore sustained in vivo efficacy), it is not sufficient. As such, the use of standard GPCR screening assays alone is insufficient for selecting compounds with sustained in vivo efficacy.

Also described herein is the discovery that in vitro measures of selectivity for the CB2 receptor (versus the CB1 receptor) translate poorly to in vivo observations in rodents, with CB2 selectivity being greatly reduced in in vivo assays compared to in vitro assays. This is potentially the result of a CB1 receptor reserve (and therefore CB1 effects with very low receptor occupancy) in the rodent central nervous system.

Finally, also described herein is the discovery that several compounds that have been investigated as clinical candidates for CB2 receptor-mediated disorders are only partial CB2 receptor agonists, demonstrate weak CB2 receptor internalization, and/or target the CB1 receptor. For example, although GW842166 (which failed to advance past phase 2 clinical trials) exhibits 99% efficacy in a β-arrestin assay, the compound was found to drive weak CB2 receptor internalization, and in vivo effects of the compound were found to be blocked by a CB1 antagonist (see Example 14).

The fact that compounds can induce CB2 receptor activation—yet insufficiently internalize the CB2 receptor—has substantial implications for the development of CB2 receptor agonists. Described herein are methods that incorporate information regarding CB2 receptor internalization to address the shortcomings of previous approaches. These methods enable the identification of compounds with profiles that are distinct from failed clinical candidates.

Knowledge of the CB2 signaling pathway suggests utility of such compounds for several clinical conditions, including pain, fibrosis, and conditions related thereto. The analgesic properties of cannabinoids have been recognized for many years. For example, animal studies have demonstrated that the CB1/CB2 receptor agonists anandamide, THC, CP55,940, and WIN 55212-2 are effective against acute and chronic pain from chemical, mechanical, and thermal pain stimuli (reviewed in Pharmacol. Ther. 95:127-135, 2002; Pharmacol. Rev. 58(3):389-462, 2006). In humans, topical administration of the CB1/CB2 receptor agonist HU-210 attenuates capsaicin-induced hyperalgesia and allodynia (Pain 102:283-288, 2003), and co-administration of the CB1/CB2 receptor agonist THC and cannabidiol (nabiximols, trademark Sativex®) provides relief from cancer-associated pain (GW Pharmaceuticals press releases dated Jan. 19, 2005; Jun. 19, 2007) and multiple-sclerosis-associated pain and spasticity (GW Pharmaceuticals press releases dated Sep. 27, 2005; Mar. 11, 2009). Further, Compound 699 disclosed herein is a potent and selective CB2 receptor agonist. Compound 699 has demonstrated efficacy in a rat model of osteoarthritis pain comparable to morphine after acute dosing, avoidance of tolerance after repeated dosing, and sustained analgesia after sub-chronic dosing. Compound 699 has also demonstrated efficacy comparable to gabapentin in paclitaxel-induced neuropathic pain after acute dosing, and efficacy in painful peripheral diabetic neuropathy in both ZDF and STZ rats.

The CB2 signaling pathway has also been identified as an anti-fibrogenic pathway (J Hepatology 59(4):891-896, 2013; Gastroenterology 128:742-755, 2005). CB2 receptors are expressed by hepatocytes in nonalcoholic fatty liver disease, but not in normal liver (Liver International 27(2):215-219, 2007). Reports have also shown upregulation of CB2 receptor expression in hepatic myofibroblasts and vascular endothelial cells (World J Gastroenterol. 28; 14(40):6109-14, Oct. 28, 2008). In addition, the endogenous cannabinoid system is highly upregulated during chronic liver disease, and experimental and clinical findings indicate that it plays a role in the pathogenesis of liver fibrosis (Liver International 33(9):1298-1308, 2013). For example, CB2-deficient mice exhibit enhanced steatosis and fibrosis, and the administration of CB2 receptor agonist JWH-133 to rats with established cirrhosis improves liver fibrosis (Liver Int 31:860-870, 2011; J Hepatology 59(4):891-896, 2013). CB2 receptor activation has also been found to decrease liver fibrosis following bile duct ligation by counteracting IL-17-induced immune and fibrogenic responses (J Hepatology 59(1):296-306, 2014). Some studies also suggest that cannabinoid receptors contribute to the pathogenesis of cardio-circulatory disturbances occurring in advanced cirrhosis, and demonstrate a regression in fibrosis following chronic stimulation of the CB2 receptor in cirrhotic rats (Liver International 33(9):1298-1308, 2013; J Pharmacol Exp Ther 324:475-483, 2008).

The CB2 receptor also plays a role in fibrotic processes outside the liver. For example, CB2-deficient mice are more sensitive to bleomycin-induced dermal fibrosis, and selective CB2 receptor agonist JWH-133 has been shown to reduce leukocyte infiltration and dermal thickening (Arthritis Rheum 60:1129-1136, 2009). The CB2 receptor has been identified as a potential target for the treatment of systemic sclerosis because it controls both skin fibroblast proliferation and the autoimmune reaction (Servettaz A, et al. (2010) Targeting the cannabinoid pathway limits the development of fibrosis and autoimmunity in a mouse model of systemic sclerosis. Am J Pathol 177:187-196). Further, CB2 receptor agonists Δ-9-tetrahydrocannabinol (THC) and cannabidiol have been found to suppress the production of IL-17 and IL-6, and boost the expression of anti-inflammatory cytokine IL-10 (J Neuroimmune Pharmacology 8(5):1265-76, 2013). In addition, pirfenidone (recently approved by the U.S. FDA for the treatment of idiopathic pulmonary fibrosis) has been found to enhance CB2 gene expression in patients with chronic hepatitis C (BMC Gastroenterology 14:1-20, 2014).

Further, a study in a model of diabetic nephropathy suggests a protective effect of signaling through the CB2 receptor (Diabetes 60:2386-2396, 2011). CB2 is expressed in chondrocytes, and cannabinoids may protect cartilage matrix from cytokine-induced degradation (J Pharmacy and Pharmacology 58:351-358). CB2 receptor agonists have been shown in several animal models to exhibit protective effects in ischemic organs, such as the liver and heart. The CB2 receptor has also been suggested as having a role in improving outcomes in chronic neuroinflammatory conditions and reducing secondary damage following acute injury (Current Neuropharmacology 5:73-80, 2007).

The CB2 receptor has emerged as a critical player in regulation of pain, inflammation, atherosclerosis, and osteoporosis, with a key role during chronic and acute liver injury (including fibrogenesis associated to chronic liver diseases, ischaemia-reperfusion-induced liver injury, and hepatic encephalopathy associated to acute liver failure) (Br J Pharmacol 153:286-289, 2008). Described herein are compounds that interact with and activate the CB2 receptor (which are also referred to herein as “CB2 receptor agonists” or “CB2 agonists”) and therefore have utility for the treatment of CB2 receptor-mediated disorders.

Examples of compounds that modulate the activity of the CB2 receptor are disclosed in PCT patent publications WO2011/025541, WO2012/116276, WO2012/116278, WO2012/116277, and WO2012/116279, and U.S. provisional patent application 62/084,165, which are each incorporated herein by reference in their entirety. Several of the compounds disclosed herein (e.g., Compounds 493, 699, 700, 704, 765, 820, 841, and 919) are also disclosed in WO2011/025541, have the same numerical identifiers as in WO2011/025541, and can be prepared as disclosed therein.

One aspect of the present invention is directed to compounds, as described herein, and pharmaceutically acceptable salts, solvates, and hydrates thereof, which increase internalization of the CB2 receptor, and uses related thereto.

Provided is a method comprising measuring internalization of CB2 receptors in a cell following contact with a compound; and formulating the compound into a pharmaceutical composition if internalization of the CB2 receptors is increased to a predefined level following contact with the compound. In some embodiments, the method further comprises administering a compound that increases internalization of CB2 receptors to the predefined level to a mammal; and measuring efficacy of the compound in the mammal. In some embodiments, the method further comprises measuring selectivity of the compound for the CB2 receptor relative to the CB1 receptor. In some embodiments, the method further comprises measuring selectivity of the compound for the human CB2 receptor relative to the human CB1 receptor.

Also provided is a method comprising selecting a compound previously identified as increasing internalization of CB2 receptors to a predefined level in a cell; and formulating the compound into a pharmaceutical composition. In some embodiments, the method further comprises administering a compound that increases internalization of CB2 receptors to the predefined level to a mammal; and measuring efficacy of the compound in the mammal. In some embodiments, the method further comprises measuring selectivity of the compound for the CB2 receptor relative to the CB1 receptor. In some embodiments, the method further comprises measuring selectivity of the compound for the human CB2 receptor relative to the human CB1 receptor.

Also provided is a method comprising selecting a compound previously identified as: increasing internalization of CB2 receptors in a cell; and demonstrating in vivo efficacy in a mammal; and formulating the compound into a pharmaceutical composition. In some embodiments, the method further comprises administering a compound that increases internalization of CB2 receptors to the predefined level to a mammal; and measuring efficacy of the compound in the mammal. In some embodiments, the method further comprises measuring selectivity of the compound for the CB2 receptor relative to the CB1 receptor. In some embodiments, the method further comprises measuring selectivity of the compound for the human CB2 receptor relative to the human CB1 receptor.

Also provided is a method comprising measuring internalization of CB2 receptors in a cell following contact with a compound; and producing, isolating, or synthesizing the compound if the compound increases internalization of the CB2 receptors to a predefined level in the cell. In some embodiments, the method further comprises administering a compound that increases internalization of CB2 receptors to the predefined level to a mammal; and measuring efficacy of the compound in the mammal. In some embodiments, the method further comprises measuring selectivity of the compound for the CB2 receptor relative to the CB1 receptor. In some embodiments, the method further comprises measuring selectivity of the compound for the human CB2 receptor relative to the human CB1 receptor.

Also provided is a method comprising measuring internalization of CB2 receptors in a cell following contact with a compound; administering a compound that increases internalization of the CB2 receptors to a predefined level to a mammal; and formulating the compound into a pharmaceutical composition if the compound demonstrates in vivo efficacy in the mammal. In some embodiments, the method further comprises measuring selectivity of the compound for the CB2 receptor relative to the CB1 receptor. In some embodiments, the method further comprises measuring selectivity of the compound for the human CB2 receptor relative to the human CB1 receptor.

Also provided is a method comprising selecting a compound identified as increasing internalization of CB2 receptors to a predefined level in a cell; and administering the compound to an individual in need thereof. In some embodiments, the method further comprises administering a compound that increases internalization of CB2 receptors to the predefined level to a mammal; and measuring efficacy of the compound in the mammal. In some embodiments, the method further comprises measuring selectivity of the compound for the CB2 receptor relative to the CB1 receptor. In some embodiments, the method further comprises measuring selectivity of the compound for the human CB2 receptor relative to the human CB1 receptor.

Also provided is a method comprising measuring internalization of CB2 receptors in a cell following contact with a compound; administering a compound that increases internalization of the CB2 receptors to a predefined level to a mammal; and formulating the compound into a pharmaceutical composition if the compound demonstrates in vivo efficacy in the mammal. In some embodiments, the method further comprises administering a compound that increases internalization of CB2 receptors to the predefined level to a mammal; and measuring efficacy of the compound in the mammal. In some embodiments, the method further comprises measuring selectivity of the compound for the CB2 receptor relative to the CB1 receptor. In some embodiments, the method further comprises measuring selectivity of the compound for the human CB2 receptor relative to the human CB1 receptor.

Also provided is a method comprising contacting a compound with a cell expressing the CB2 receptor; measuring internalization of the CB2 receptor in the cell following the contact; and formulating the compound if the compound increases internalization of the CB2 receptor to a predefined level in the cell. In some embodiments, the method further comprises measuring agonism of the compound for the CB2 receptor. In some embodiments, the method further comprises measuring selectivity of the compound for the human CB2 receptor relative to the human CB1 receptor. In some embodiments, the method further comprises measuring efficacy of the compound in vivo.

Also provided is a method comprising contacting a compound with a cell expressing the CB2 receptor; measuring internalization of the CB2 receptor in the cell following the contact; and administering the compound to an individual in need thereof if the compound increases internalization of the CB2 receptor to a predefined level in the cell. In some embodiments, the method further comprises measuring agonism of the compound for the CB2 receptor. In some embodiments, the method further comprises measuring selectivity of the compound for the human CB2 receptor relative to the human CB1 receptor. In some embodiments, the method further comprises measuring efficacy of the compound in vivo.

Also provided is a method comprising formulating a compound into a pharmaceutical composition, wherein the compound has previously been identified as increasing internalization of the CB2 receptor to a predefined level in a cell.

In some embodiments, the method further comprises measuring agonism of the compound for the CB2 receptor.

In some embodiments, the method further comprises measuring selectivity of the compound for the CB2 receptor relative to the CB1 receptor.

In some embodiments, the method further comprises measuring selectivity of the compound for the human CB2 receptor relative to the human CB1 receptor.

In some embodiments, the method further comprises measuring efficacy of the compound in vivo.

In some embodiments, the method further comprises administering the pharmaceutical composition to an individual in need thereof.

In some embodiments, the methods described herein further comprise selecting a compound based on brain penetration. In some embodiments, the methods described herein further comprise measuring brain penetration for a compound. In some embodiments, brain penetration is measured in an in vitro assay. For example, in some embodiments, an endothelial cell culture model of the blood-brain barrier (BBB) is assessed. In some embodiments, brain penetration is measured in silico. In some embodiments, brain penetration is measured in a non-human mammal. In some embodiments, brain penetration is measured in a human. In some embodiments, compounds and/or pharmaceutical compositions with low brain penetration are selected. For example, in some embodiments, compounds and/or pharmaceutical compositions with low brain penetration are selected to enhance selectivity for the CB2 receptor.

In some embodiments, the compound has previously been identified as exhibiting at least 500-fold, at least 750-fold, at least 1000-fold, at least 2000-fold, at least 3000-fold, at least 4000-fold, at least 5000-fold, at least 6000-fold, at least 7000-fold, at least 8000-fold, at least 9000-fold, or at least 10,000-fold selectivity for the human CB2 receptor relative to the human CB1 receptor.

In some embodiments, the compound in the pharmaceutical composition is in an amount sufficient for the treatment or prevention of a CB2 receptor-mediated disorder.

In some embodiments, the compound is in an amount sufficient for the treatment or prevention of a CB2 receptor-mediated disorder.

In some embodiments, the ability of a compound to increase internalization of the CB2 receptors to the predefined level is indicative of the compound being useful for the treatment of a CB2 receptor-mediated disorder.

In some embodiments, the compound is suitable for the treatment of a CB2 receptor-mediated disorder.

In some embodiments, the CB2 receptor-mediated disorder is pain, fibrosis, or a condition related thereto.

In some embodiments, the CB2 receptor-mediated disorder is selected from: pain associated with osteoarthritis, neuropathic pain, acute post-operative pain, liver fibrosis, primary biliary cirrhosis, nonalcoholic steatohepatitis, renal fibrosis, endometriosis, and interstitial cystitis.

In some embodiments, the mammal is a non-human mammal.

In some embodiments, the CB2 receptors are human CB2 receptors.

In some embodiments, the CB2 receptors are recombinant.

In some embodiments, the CB2 receptors comprise a sequence with at least 75%, at least 80%, at least 85%, at least 90%, at least 95%, at least 96%, at least 97%, at least 98%, at least 99%, or 100% identity to NCBI Reference Sequence NP_001157614.1.

In some embodiments, the CB2 receptors are encoded by a nucleotide sequence comprising at least 75%, at least 80%, at least 85%, at least 90%, at least 95%, at least 96%, at least 97%, at least 98%, at least 99%, or 100% identity to NCBI Reference Sequence NM_001841.

In some embodiments, the CB2 receptors comprise a sequence with at least 75%, at least 80%, at least 85%, at least 90%, at least 95%, at least 96%, at least 97%, at least 98%, at least 99%, or 100% identity to NCBI Reference Sequence NP_001832.1.

In some embodiments, the compound is non-naturally occurring.

In some embodiments, the compound is orally active.

In some embodiments, the cell is a liver cell, a kidney cell, or a lung cell.

In some embodiments, internalization is measured, or has been measured, relative to the level of internalization that would occur if the cell were contacted with a full CB2 receptor internalization agonist.

In some embodiments, internalization is measured, or has been measured, relative to the level of internalization that would occur if the cell were contacted with CP55,940.

In some embodiments, the compound increases internalization of the CB2 receptors to at least 75%, at least 80%, at least 85%, at least 90%, at least 95%, at least 96%, at least 97%, at least 98%, or at least about 99% the level of internalization that would occur if the cell were contacted with CP55,940.

In some embodiments, internalization is measured using a method selected from: flow cytometry, fluorescence microscopy, and enzyme complementation.

In some embodiments, efficacy of the compound in the mammal is measured at least about 2 hours, at least about 3 hours, at least about 4 hours, at least about 5 hours, or at least about 6 hours following administration to the mammal.

In some embodiments, the mammal is an animal model for pain, fibrosis, or a condition related thereto.

Also provided are pharmaceutical compositions prepared according to the methods described herein.

Also provided are compounds identified as increasing internalization to a predefined level according to the methods described herein.

Also provided are pharmaceutical compositions comprising a compound described herein.

Also provided are pharmaceutical compositions comprising a compound described herein and a pharmaceutical excipient.

In some embodiments, the pharmaceutical compositions are suitable for oral, rectal, nasal, topical, buccal, sub-lingual, or vaginal, or in a form suitable for administration by inhalation, insufflation, or by a transdermal patch.

In some embodiments, the pharmaceutical compositions are suitable for oral administration.

Also provided is a process for preparing a pharmaceutical composition, comprising admixing a pharmaceutically acceptable carrier with a compound selected as increasing internalization of CB2 receptors in a cell to at least 75%, at least 80%, at least 85%, at least 90%, at least 95%, at least 96%, at least 97%, at least 98%, or at least about 99% the level of internalization that would occur if the cell were contacted with CP55,940. In some embodiments, the compound is in an amount sufficient for the treatment or prevention of a CB2 receptor-mediated disorder. In some embodiments, the CB2 receptor-mediated disorder is pain, fibrosis, or a condition related thereto.

Also provided is a process for preparing a pharmaceutical composition, comprising admixing a pharmaceutically acceptable carrier with a compound selected as increasing internalization of CB2 receptors in a cell to at least 75%, at least 80%, at least 85%, at least 90%, at least 95%, or at least about 99% the level of internalization that would occur if the cell were contacted with CP55,940, wherein the compound has also been selected as exhibiting at least 50-fold, at least 100-fold, at least 500-fold, at least 750-fold, at least 1000-fold, at least 5000-fold, or at least 10,000-fold selectivity for the human CB2 receptor relative to the human CB1 receptor. In some embodiments, the compound is in an amount sufficient for the treatment or prevention of a CB2 receptor-mediated disorder. In some embodiments, the CB2 receptor-mediated disorder is pain, fibrosis, or a condition related thereto.

Also provided is the use of a compound selected as increasing internalization of CB2 receptors in a cell to at least 75%, at least 80%, at least 85%, at least 90%, at least 95%, or at least about 99% the level of internalization of CB2 receptors following contact with CP55,940 in the manufacture of a medicament for treating or preventing a CB2 receptor-mediated disorder in an individual. In some embodiments, the CB2 receptor-mediated disorder is pain, fibrosis, or a condition related thereto.

Also provided is the use of a compound selected as increasing internalization of CB2 receptors in a cell to at least 75%, at least 80%, at least 85%, at least 90%, at least 95%, or at least about 99% the level of internalization that would occur following contact with CP55,940, wherein the compound has been also selected as exhibiting at least 50-fold, at least 100-fold, at least 500-fold, at least 750-fold, at least 1000-fold, at least 5000-fold, or at least 10,000-fold selectivity for the human CB2 receptor relative to the human CB1 receptor, in the manufacture of a medicament for treating or preventing a CB2 receptor-mediated disorder in an individual. In some embodiments, the CB2 receptor-mediated disorder is pain, fibrosis, or a condition related thereto.

Also provided is a compound for use in the treatment of a CB2 receptor-mediated disorder in an individual, wherein the compound has been selected as increasing internalization of CB2 receptors to at least 75%, at least 80%, at least 85%, at least 90%, at least 95%, or at least about 99% the level of internalization that would occur following contact with CP55,940, with a pharmaceutically acceptable carrier. In some embodiments, the CB2 receptor-mediated disorder is pain, fibrosis, or a condition related thereto.

Also provided is a compound for use in the treatment of a CB2 receptor-mediated disorder in an individual, wherein the compound has been selected as increasing internalization of CB2 receptors to at least 75%, at least 80%, at least 85%, at least 90%, at least 95%, or at least about 99% the level of internalization that would occur following contact with CP55,940, and wherein the compound has also been selected as exhibiting at least 50-fold, at least 100-fold, at least 500-fold, at least 1000-fold, at least 5000-fold, or at least 10,000-fold selectivity for the human CB2 receptor relative to the human CB1 receptor, with a pharmaceutically acceptable carrier. In some embodiments, the CB2 receptor-mediated disorder is pain, fibrosis, or a condition related thereto.

Also provided are methods for identifying compounds that drive CB2 receptor internalization.

### BRIEF DESCRIPTION OF FIGURES

FIGS. 1A-1C show measurements from β-arrestin, receptor internalization, and in vivo assays for the CB2 receptor following the administration of several compounds disclosed herein.

FIGS. 2A-2B show rapid loss of in vivo efficacy despite high plasma concentrations of Compound 820.

FIGS. 3A-3B show rapid loss of in vivo efficacy despite increasing plasma concentrations of Compound 704.

FIG. 4 shows rapid loss of in vivo efficacy following administration of Compound 493.

FIG. 5 shows rapid loss of in vivo efficacy following administration of Compound 700.

FIGS. 6A-6B show sustained in vivo efficacy despite decreasing plasma concentrations of Compound 699.

FIGS. 7A-7B show sustained in vivo efficacy despite decreasing plasma concentrations of Compound 919.

FIGS. 8A-8B show sustained in vivo efficacy despite decreasing plasma concentrations of Compound 765.

FIGS. 9A-9B show sustained in vivo efficacy despite decreasing plasma concentrations following administration of Compound 841.

FIG. 10 shows a comparison of CB2 receptor internalization following administration of Compound 493 and Compound 841.

FIG. 11 shows a summary of the efficacies of CB2 receptor agonists with potencies less than 500 nM in β-arrestin assays, including Eli Lilly Compound LY2828360 as an example.

### DETAILED DESCRIPTION

As used in the present specification, the following words and phrases are generally intended to have the meanings as set forth below, except to the extent that the context in which they are used indicates otherwise.

The term “adverse event” refers to any untoward medical occurrence that may present itself during treatment. Adverse events associated with treatment may include, for example, psychotropic effects. In the methods disclosed herein, the term “adverse event” can be replaced by other more general terms such as “toxicity.” The term “reducing the risk” of an adverse event means reducing the probability that an adverse event or toxic event will occur.

The term “agonist” refers to a moiety that interacts with and activates a G-protein-coupled receptor, for instance a CB2 receptor, and can thereby initiates a physiological or pharmacological response characteristic of that receptor. For example, an agonist may activate an intracellular response upon binding to a receptor, or enhance GTP binding to a membrane.

The term “combination” as used in reference to drug combinations and/or combination of a compound or a pharmaceutically acceptable salt, solvate, or hydrate thereof with at least one supplemental agent refers to (1) a product comprised of two or more components, i.e., drug/device, biologic/device, drug/biologic, or drug/device/biologic, that are physically, chemically, or otherwise combined or mixed and produced as a single entity; (2) two or more separate products packaged together in a single package or as a unit and comprised of drug and device products, device and biological products, or biological and drug products; (3) a drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (4) any investigational drug, device, or biological product packaged separately that according to its proposed labeling is for use only with another individually specified investigational drug, device, or biological product where both are required to achieve the intended use, indication, or effect. Combinations include without limitation a fixed-dose combination product (FDC) in which two or more separate drug components are combined in a single dosage form; a co-packaged product comprising two or more separate drug products in their final dosage forms, packaged together with appropriate labeling to support the combination use; and an adjunctive therapy in which a patient is maintained on a second drug product that is used together with (i.e., in adjunct to) the primary treatment, although the relative doses are not fixed, and drugs or biologics that are not necessarily given at the same time. Adjunctive therapy products may be co-packaged, and may or may not be labeled for concomitant use.

The term “composition” refers to a compound or crystalline form thereof, including but not limited to, salts, solvates, and hydrates of a compound of the present invention, in combination with at least one additional component, such as, a composition obtained/prepared during synthesis, preformulation, in-process testing (i.e., TLC, HPLC, NMR samples), and the like.

The term “compound described herein” refers to a compound explicitly recited herein, or a compound identified according to a method described herein.

The term “fold” (e.g., 10-fold) is used herein interchangeably with a value followed by “X” (e.g., 10×) or “times” (e.g., 10 times).

The term “fibrosis” can be used interchangeably with “fibrotic disease,” “fibrotic disorder,” and/or “fibrotic condition.”

The term “greater than” can be used interchangeably with the symbol > and the term “less than” is used interchangeably with the symbol <. Likewise, the term “greater than or equal to” is used interchangeably with the symbol ≥, and the term “less than or equal to” is used interchangeably with the symbol ≤.

The term “hydrate” refers to a compound of the invention or a salt thereof that further includes a stoichiometric or non-stoichiometric amount of water bound by non-covalent intermolecular forces.

The term “in need of treatment” and the term “in need thereof” when referring to treatment can be used interchangeably to mean a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit, or ameliorate the disease, condition, or disorder.

The term “individual” refers to a mammal, such as a mouse, rat, other rodent, rabbit, dog, cat, pig, cow, sheep, horse, non-human primate, or human. In some embodiments, “individual” refers to a non-human mammal. In some embodiments, “individual” refers to a human.

The term “modulate or modulating” refers to an increase or decrease in the amount, quality, response or effect of a particular activity, function or molecule.

The term “pharmaceutical composition” refers to a specific composition comprising at least one active ingredient; including but not limited to, salts, solvates, and hydrates of compounds of the present invention, whereby the composition is amenable to investigation for a specified, efficacious outcome in a mammal (for example, without limitation, a human). Those of skill in the art will understand and appreciate the techniques appropriate for determining whether an active ingredient has a desired efficacious outcome based upon the needs of one of skill in the art.

The phrase “pharmaceutically acceptable salts, solvates, and hydrates” when referring to a compound/compounds as described herein embraces pharmaceutically acceptable solvates and/or hydrates of the compound/compounds, pharmaceutically acceptable salts of the compound/compounds, as well as pharmaceutically acceptable solvates and/or hydrates of pharmaceutically acceptable salts of the compound/compounds. It is also understood that when the phrase “pharmaceutically acceptable solvates and hydrates” or the phrase “pharmaceutically acceptable solvate or hydrate” is used when referring to a compound/compounds as described herein that are salts, it embraces pharmaceutically acceptable solvates and/or hydrates of such salts. It is also understood by a person of skill in the art that hydrates are a subgenus of solvates.

The terms “prevent,” “preventing,” and “prevention” refer to the elimination or reduction of the occurrence or onset of one or more symptoms associated with a particular disorder. For example, the terms “prevent,” “preventing,” and “prevention” can refer to the administration of therapy on a prophylactic or preventative basis to an individual who may ultimately manifest at least one symptom of a disorder but who has not yet done so. Such individuals can be identified on the basis of risk factors that are known to correlate with the subsequent occurrence of the disease, such as the presence of a biomarker. Alternatively, prevention therapy can be administered as a prophylactic measure without prior identification of a risk factor. Delaying the onset of the at least one episode and/or symptom of a disorder can also be considered prevention or prophylaxis.

The term “solvate” refers to a compound of the invention or a salt thereof that further includes a stoichiometric or non-stoichiometric amount of a solvent bound by non-covalent intermolecular forces. Preferred solvents are volatile, non-toxic, and/or acceptable for administration to humans in trace amounts.

The term “supplemental agent” refers to an additional therapeutic agent which complements the activity of a compound or its pharmaceutically acceptable salt, solvate, or hydrate thereof described herein.

The term “therapeutically effective amount” refers to the amount of active compound or pharmaceutical agent that elicits the biological or medicinal response in a tissue, system, animal, or human that is being sought by an individual, researcher, veterinarian, medical doctor, or other clinician or caregiver, which can include one or more of the following:

(1) preventing the disorder, for example, preventing a disease, condition, or disorder in an individual who may be predisposed to the disease, condition, or disorder but does not yet experience or display the relevant pathology or symptomatology;

(2) inhibiting the disorder, for example, inhibiting a disease, condition, or disorder in an individual who is experiencing or displaying the relevant pathology or symptomatology (i.e., arresting further development of the pathology and/or symptomatology); and

(3) ameliorating the disorder, for example, ameliorating a disease, condition, or disorder in an individual who is experiencing or displaying the relevant pathology or symptomatology (i.e., reversing the pathology and/or symptomatology).

The terms “treat,” “treating,” and “treatment” refer to the administration of therapy to an individual who already manifests, or who has previously manifested, at least one symptom of a disease, disorder, or condition. For example, “treating” can include any of the following with respect to a disease, disorder, condition, dependence, or behavior: alleviating, abating, ameliorating, improving, inhibiting (e.g., arresting the development), relieving, or causing regression. “Treating” can also include treating the symptoms, preventing additional symptoms, preventing the underlying physiological causes of the symptoms, or stopping the symptoms (either prophylactically and/or therapeutically) of a disease, disorder, or condition. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

When an integer is used in a method disclosed herein, the term “about” can be inserted before the integer. For example, the term “greater than 10 mg” can be substituted with “greater than about 10 mg.”

Throughout this specification, unless the context requires otherwise, the word “comprise,” or variations such as “comprises” or “comprising” will be understood to imply the inclusion of a stated step or element or integer or group of steps or elements or integers, but not the exclusion of any other step or element or integer or group of elements or integers.

### Indications and Methods of Treatment

In addition to the disorders described above, the compounds described herein are useful in the treatment or prevention of several other disorders and/or the amelioration of symptoms thereof.

One aspect of the present invention relates to methods of identifying compounds useful for the treatment or prevention of a CB2 receptor-mediated disorder. In some embodiments, the CB2 receptor-mediated disorder is one or more of the disorders described herein.

Another aspect of the present invention relates to methods of selecting compounds useful for the treatment or prevention of a CB2 receptor-mediated disorder. In some embodiments, the CB2 receptor-mediated disorder is one or more of the disorders described herein.

Another aspect of the present invention relates to the production, isolation, or synthesis of a compound identified as described herein. In some embodiments, the compound is in an amount sufficient for the treatment of a particular CB2 receptor-mediated disorder. In some embodiments, the CB2 receptor-mediated disorder is one or more of the disorders described herein.

Another aspect of the present invention relates to the preparation of pharmaceutical compositions, comprising admixing a pharmaceutically acceptable carrier with a compound described herein. In some embodiments, the pharmaceutical composition is in an amount sufficient for the treatment of a particular CB2 receptor-mediated disorder. In some embodiments, the CB2 receptor-mediated disorder is one or more of the disorders described herein.

Another aspect of the present invention relates to compounds as described herein, for use in a method of treatment of the human or animal body by therapy.

Another aspect of the present invention relates to the use of compounds described herein in the treatment or prevention of a CB2 receptor-mediated disorder. In some embodiments, the CB2 receptor-mediated disorder is one or more of the disorders described herein.

Another aspect of the present invention relates to compounds described herein for use in a method of treatment or prevention of a CB2 receptor-mediated disorder. In some embodiments, the CB2 receptor-mediated disorder is one or more of the disorders described herein.

Another aspect of the present invention relates to the use of compositions described herein in the manufacture of a medicament for treating or preventing a CB2 receptor-mediated disorder. In some embodiments, the CB2 receptor-mediated disorder is one or more of the disorders described herein.

Another aspect of the present invention relates to methods for the treatment or prevention of a CB2 receptor-mediated disorder in an individual, comprising administering to the individual in need thereof a therapeutically effective amount of a compound as described herein. In some embodiments, the CB2 receptor-mediated disorder is one or more of the disorders described herein.

Another aspect of the present invention relates to pharmaceutical compositions as described herein, for use in a method of treatment of the human or animal body by therapy.

Another aspect of the present invention relates to the use of pharmaceutical compositions described herein in the treatment or prevention of a CB2 receptor-mediated disorder. In some embodiments, the CB2 receptor-mediated disorder is one or more of the disorders described herein.

Another aspect of the present invention relates to pharmaceutical compositions described herein for use in a method of treatment or prevention of a CB2 receptor-mediated disorder. In some embodiments, the CB2 receptor-mediated disorder is one or more of the disorders described herein.

Another aspect of the present invention relates to the use of compositions described herein in the manufacture of a medicament for treating or preventing a CB2 receptor-mediated disorder. In some embodiments, the CB2 receptor-mediated disorder is one or more of the disorders described herein.

Another aspect of the present invention relates to methods for the treatment or prevention of a CB2 receptor-mediated disorder in an individual, comprising administering to the individual in need thereof a therapeutically effective amount of a pharmaceutical composition as described herein. In some embodiments, the CB2 receptor-mediated disorder is one or more of the disorders described herein.

Without limitation, additional disorders include the following CB2 receptor-mediated disorders.

### I. Pain

In some embodiments, the CB2 receptor-mediated disorder is pain or a condition related thereto. As discussed herein, the CB2 receptor plays a role in mediating the analgesic effects of cannabinoids (reviewed in Br. J. Pharmacol. 153:319-334, 2008). For example, systemic delivery of the CB2-selective agonist AM1241 suppresses hyperalgesia induced in the carrageenan, capsaicin, and formalin models of inflammatory pain in rodents (reviewed in Br. J. Pharmacol. 153:319-334, 2008). Local (subcutaneous) or systemic administration of AM1241 also reverses tactile and thermal hypersensitivity in rats following ligation of spinal nerves in the chronic constriction injury model of neuropathic pain (Pain 93:239-245, 2001; PNAS 100(18):10529-10533, 2003), an effect which is inhibited by treatment with the CB2-selective antagonist AM630 (PNAS 102(8):3093-8, 2005). The CB2-selective agonist GW405833 administered systemically significantly reverses hypersensitivity to mechanical stimuli in rats following ligation of spinal nerves (Pain 143:206-212, 2009). Thus, CB2 receptor agonists have also been shown to attenuate pain in experimental models of acute, inflammatory, and neuropathic pain, and hyperalgesia.

Accordingly, CB2 agonists find use in the treatment and/or prophylaxis of acute nociception and inflammatory hyperalgesia, as well as the allodynia and hyperalgesia produced by neuropathic pain. For example, these agonists are useful as an analgesic to treat pain arising from autoimmune conditions; allergic reactions; bone and joint pain; muscle pain; dental pain; nephritic syndrome; scleroderma; thyroiditis; migraine and other headache pain; pain associated with diabetic neuropathy; fibromyalgia, HIV-related neuropathy, sciatica, and neuralgias; pain arising from cancer; and pain that occurs as an adverse effect of therapeutics for the treatment of disease.

Another aspect of the present invention relates to compounds and pharmaceutical compositions useful for the treatment of pain in an individual. Some embodiments relate to the treatment of pain associated with osteoarthritis in an individual, comprising administering to the individual in need thereof, a therapeutically effective amount of a pharmaceutical composition as described herein. Some embodiments relate to the treatment of neuropathic pain in an individual, comprising administering to the individual in need thereof, a therapeutically effective amount of a pharmaceutical composition as described herein. Some embodiments relate to the treatment of acute post-operative pain in an individual, comprising administering to the individual in need thereof, a therapeutically effective amount of a pharmaceutical composition as described herein.

Another aspect of the present invention relates to the use of a pharmaceutical composition as described herein, in the treatment of pain. Some embodiments relate to the use of a pharmaceutical composition as described herein, in the treatment of pain associated with osteoarthritis. Some embodiments relate to the use of a pharmaceutical composition as described herein, in the treatment of neuropathic pain. Some embodiments relate to the use of a pharmaceutical composition as described herein, in the treatment of acute post-operative pain.

Another aspect of the present invention relates to pharmaceutical compositions as described herein, for use in a method of treatment of pain. Some embodiments relate to pharmaceutical compositions as described herein, for use in a method of treatment of pain associated with osteoarthritis. Some embodiments relate to pharmaceutical compositions as described herein, for use in a method of treatment of neuropathic pain. Some embodiments relate to pharmaceutical compositions as described herein, for use in a method of treatment of acute post-operative pain.

### II. Disorders of the Immune System

IIa. Autoimmune Disorders.

In some embodiments, the CB2 receptor-mediated disorder is an autoimmune disorder. Cannabinoid receptor agonists have been shown to attenuate aberrant immune responses in autoimmune disorders, and in some cases, to provide protection to the tissue that is being inappropriately targeted by the immune system. For example, multiple sclerosis (MS) is an autoimmune disorder that results in the demyelination of neurons in the CNS. The CB1/CB2 receptor agonist THC significantly inhibits the severity of clinical disease in the Experimental Autoimmune Encephalomyelitis (EAE) mouse model of MS, an effect that is believed to be mediated by CB1 on neurons and CB2 on immune cells (Nat. Med. 13(4):492-497, 2007). Consistent with these results, CB2-selective agonist HU-308 markedly reduces the recruitment of immature myeloid cells and T cells, microglial and infiltrating myeloid cell proliferation, and axonal loss in the EAE model (J. Biol. Chem. 283(19):13320-9, 2008). Likewise, the CB1/CB2 receptor agonist WIN 55212-2 significantly inhibits leukocyte rolling and adhesion in the brain in the EAE mouse model, an effect that is blocked by the CB2-selective antagonist SR144528 but not the CB1-selective antagonist SR141716A (Mult. Sclerosis 10(2):158-64, 2004). Accordingly, CB2 receptor agonists find use in the treatment and/or prophylaxis of multiple sclerosis and related autoimmune demyelinating diseases, e.g. Guillan-Barré syndrome, polyradiculoneuropathy, and chronic inflammatory demyelination.

As another example, the autoimmune disease rheumatoid arthritis (RA) is a chronic, systemic inflammatory disorder of the skeletal system that principally attacks the joints to produce an inflammatory synovitis and that often progresses to destruction of the articular cartilage and ankylosis of the joints. The CB1/CB2 receptor agonists WIN 55212-2 and HU-210 significantly inhibit IL-1alpha-stimulated proteoglycan and collagen degradation in bovine nasal cartilage explants in vitro (J. Pharm. and Pharmacol. 58:351-358, 2006). Accordingly, CB2 receptor agonists find use in the treatment and/or prophylaxis of autoimmune arthritic diseases, for example, rheumatoid arthritis, psoriatic arthritis, ankylosing spondylarthritis, and reactive arthritis.

IIb. Type 1 Hypersensitivity and Allergic Response.

In some embodiments, the CB2 receptor-mediated disorder is a type 1 hypersensitivity or allergic response. Cannabinoid receptor agonists have also been shown to attenuate aberrant immune responses in allergic reactions. In type-1 (or immediate) hypersensitivity, plasma cells that have been activated by an allergen secrete IgE antibodies, which bind to Fc receptors on the surface of tissue mast cells and blood basophils and eosinophils. Repeated exposure to the same allergen results in cross-linking of the bound IgE on sensitized cells, resulting in the secretion of pharmacologically active mediators such as histamine, leukotriene and prostaglandin. These mediators are responsible for the symptoms associated with allergies, including vasodilation and increased permeability, smooth muscle spasms, and leukocyte extravasation. Topical administration of the CB1/CB2 receptor agonist HU-210 reduces these histamine-induced responses in human skin (Inflamm. Res. 52:238-245, 2003). Similarly, subcutaneous injection of CB1/CB2 receptor agonist THC or increased levels of endogenous cannabinoids reduces cutaneous inflammation and the pruritus (itch) associated with it in a mouse model for allergic contact dermatitis. (Science, 316(5830), 1494-1497, 2007). Accordingly, CB2 receptor agonists find use in the treatment of allergic reactions including atopic dermatitis (pruritus/itch), urticaria (hives), asthma, conjunctivitis, allergic rhinitis (hay fever), and anaphylaxis.

IIc. Conditions Associated with CNS Inflammation.

In some embodiments, the CB2 receptor-mediated disorder is a condition associated with CNS inflammation. CB2 receptor agonists have been shown to attenuate inflammation in the CNS. For example, the administration of CB2 receptor agonists prevents the activation of microglia in rodent models of Alzheimer's Disease (Curr. Neuropharmacol. 5(2):73-80, 2007). Likewise, the administration of CB2 receptor agonists reduces the volume of infarcts by 30% in a rodent occlusion model of stroke (J. Cereb. Blood Flow Metab. 27:1387-96, 2007). Thus, CB2 receptor agonists find use in the treatment and/or prophylaxis of neuropathologies associated with CNS inflammation, e.g. Alzheimer's, stroke-induced damage, dementia, ALS, and HIV.

IId. Conditions Associated with Vascular Inflammation.

In some embodiments, the CB2 receptor-mediated disorder is a condition associated with vascular inflammation. CB2 is expressed in macrophages and T cells in atherosclerotic plaques, and the CB1/CB2 receptor agonist THC reduces the progression of atherosclerosis in ApoE knockout mice, a well-studied mouse model of atherosclerosis. The CB2-specific antagonist SR144528 completely blocks this effect in vitro and in vivo (Nature 434:782-786, 2005). Thus, CB2 receptor agonists find use in treating atherosclerosis.

IIe. Other Disorders Associated with Aberrant or Unwanted Immune Response.

In some embodiments, the CB2 receptor-mediated disorder is a disorder associated with aberrant or unwanted immune response. Given the expression of CB2 on a number of different types of immune cells and the attenuating effects that CB2 receptor agonists have been observed to have on the activities of these cells, CB2 receptor agonists are useful for the treatment and/or prophylaxis of other disorders wherein undesired immune cell activity and/or inflammation is observed. Such exemplary disorders include osteoarthritis, anaphylaxis, Behcet's disease, graft rejection, vasculitis, gout, spondylitis, viral and bacterial diseases, e.g. AIDS, and meningitis; and other autoimmune disorders such as lupus, e.g. systemic lupus erythematosus; inflammatory bowel disease, e.g. Crohn's disease, ulcerative colitis; psoriasis; autoimmune hepatitis; and type 1 diabetes mellitus.

### III. Bone and Joint Diseases

IIIa. Osteoporosis.

In some embodiments, the CB2 receptor-mediated disorder is osteoporosis. CB2 is expressed in osteoblasts, osteocytes, and osteoclasts. Osteoblasts make new bone, whereas osteoclasts degrade it. The CB2-specific agonist HU-308 enhances endocortical osteoblast numbers and activity while simultaneously inhibiting proliferation of osteoclast precursors in bone marrow-derived osteoblasts/stromal cells in vitro, and attenuates ovariectomy-induced bone loss and stimulates cortical thickness by stimulating endocortical bone formation and suppressing osteoclast number in vivo (PNAS 103(3):696-701, 2006). Thus, CB2 receptor agonists are useful for the treatment and/or prophylaxis of disease wherein bone density is decreased, such as osteoporosis.

IIIb. Arthritis.

In some embodiments, the CB2 receptor-mediated disorder is arthritis. As discussed herein, CB2 receptor agonists are useful for the treatment and/or prophylaxis of autoimmune arthritic diseases, for example, rheumatoid arthritis, psoriatic arthritis, ankylosing spondylarthritis, and reactive arthritis, and for the treatment and/or prophylaxis of inflammation associated with osteoarthritis.

### IV. Eye Disease

In some embodiments, the CB2 receptor-mediated disorder is an eye disease. Retinal pigment epithelial (RPE) cells provide trophic support to photoreceptor cells in the eye, and RPE cell death has been shown to be a major contributor to age-related macular degeneration (AMD). The CB1/CB2 receptor agonist CP55,940 significantly protects RPE cells from oxidative damage, and the CB2 receptor agonist JWH015 provides comparable protection (Mol. Vis. 15:1243-51, 2009). Accordingly, CB2 receptor agonists find use in preventing the onset or progression of vision loss associated with AMD.

### V. Cough

In some embodiments, the CB2 receptor-mediated disorder is cough. The cough reflex is predominantly under the control of two classes of sensory afferent nerve fibers, the myelinated A-delta fibers and the non-myelinated C-fibers, the activation of which (i.e. depolarization) elicits cough via the vagus nerve afferent pathway. The CB1/CB2 receptor agonist CP55,940 reduces capsaicin-, PGE2-, and hypertonic saline-induced depolarization of guinea pig and human vagus nerve preparations in vitro (British J. Pharma. 140:261-8, 2003). The CB2-selective agonist JWH133 also reduces capsaicin-, PGE2-, and hypertonic saline-induced depolarization of guinea pig and human vagus nerve preparations in vitro, and administration of CB2-selective agonist JWH133 prior to exposure to the tussive agent citric acid significantly reduces cough in conscious guinea-pigs (British J. Pharma. 140:261-8, 2003). The CB1/CB2 receptor agonists WIN 55212-2 produces a dose-dependent inhibition of the number of capsaicin-induced coughs in mice (Eur. J. Pharmacol. 474:269-272, 2003). The CB1/CB2 receptor agonist anandamide produces a dose-dependent inhibition of the number of capsaicin-induced coughs in guinea pigs (Nature 408:96-101, 2000). Thus, the CB2 receptor plays an important role in mediating the antitussive effect of cannabinoids, and CB2 receptor agonists are useful in the treatment and/or prophylaxis of cough.

### VI. Cancer

In some embodiments, the CB2 receptor-mediated disorder is cancer. A number of human leukemia and lymphoma cell lines, including Jurkat, Molt-4 and Sup-T1, express CB2 receptors and not CB1 receptors, and agonists of the CB2 receptor induce apoptosis in these and primary acute lymphoblastic leukemia (ALL) cells (US2004/0259936). Similarly, the CB2 receptor is expressed on glioblastoma cell lines and treatment with agonists of CB2 induces apoptosis of these cells in vitro (J. Neurosci. Res. 86(14):3212-20, 2008). Accordingly, CB2 receptor agonists are useful in attenuating the growth of a malignancy of the immune system, for example, leukemias, lymphomas, and solid tumors of the glial lineage.

As discussed herein, CB1/CB2 receptor agonists are also useful in providing relief from pain associated with cancer (GW Pharmaceuticals press releases dated Jan. 19, 2005; Jun. 19, 2007).

CB2-mediated signaling is involved in the in vivo and in vitro growth inhibition of prostate cancer cells, which suggests that CB2 receptor agonists have potential therapeutic interest in the management of prostate cancer. (British Journal of Cancer advance online publication 18 Aug. 2009; doi: 10.1038/sj.bjc. 6605248).

### VII. Regenerative Medicine

In some embodiments, the CB2 receptor-mediated disorder is a degenerative disorder. Agonists of CB2 modulate the expansion of the progenitor pool of neurons in the CNS. CB2 antagonists inhibit the proliferation of cultured neural stem cells and the proliferation of progenitor cells in the SVZ of young animals, whereas CB2-selective agonists stimulate progenitor cell proliferation in vivo, with this effect being more pronounced in older animals (Mol. Cell Neurosci. 38(4):526-36, 2008). Thus, agonists of CB2 are useful in regenerative medicine, for example to promote the expansion of progenitor cells for the replacement of neurons lost during injury or disease, such as Alzheimer's Disease, stroke-induced damage, dementia, amyotrophic lateral sclerosis (ALS) and Parkinson's Disease.

### VIII. Fibrosis

In some embodiments, the CB2 receptor-mediated disorder is fibrosis or a condition related thereto. Fibrosis is the accumulation of excess extracellular matrix components in organs and/or tissues. Pirfenidone was recently approved by the U.S. FDA for the treatment of idiopathic pulmonary fibrosis. However, very few treatments exist for other fibrotic conditions. There is a serious unmet need for such treatments.

As discussed herein, the CB2 signaling pathway has been identified as an anti-fibrogenic pathway. CB2 receptor agonists are useful for the treatment or prevention of fibrosis. In some embodiments, the compounds and/or pharmaceutical compositions described herein are useful for the treatment or prevention of fibrosis or condition related thereto. In some embodiments, the compounds/agonists described herein are useful for the treatment or prevention of fibrosis associated with a disease, disorder, and/or condition.

In some embodiments, the fibrosis is a chronic fibroproliferative disease. In some embodiments, the fibrosis occurs systemically. For example, in some embodiments, the fibrosis is systemic sclerosis, cystic fibrosis, nephrogenic systemic fibrosis, chronic graft versus host disease, or atherosclerosis. In some embodiments, the fibrosis is isolated to a particular organ or tissue.

In some embodiments, the fibrosis is scleroderma. In some embodiments, the fibrosis is limited scleroderma. In some embodiments, the fibrosis is limited cutaneous scleroderma. In some embodiments, the fibrosis is diffuse scleroderma. In some embodiments, the fibrosis is diffuse cutaneous scleroderma.

In some embodiments, the fibrosis occurs in the liver. In some embodiments, the fibrosis is associated with nonalcoholic steatohepatitis (NASH), alcoholic steatohepatitis (ASH), idiopathic portal hypertension, hepatic fibrosis (including congenital hepatic fibrosis), viral hepatitis B or C, autoimmune hepatitis, primary sclerosing cholangitis, primary biliary cirrhosis, or idiopathic portal hypertension. In some embodiments, the fibrosis is associated with liver steatosis. In some embodiments, the fibrosis is liver fibrosis (or “hepatic fibrosis”). In some embodiments, the fibrosis is cirrhosis. In some embodiments, the fibrosis is associated with alcoholic liver disease.

In some embodiments, the fibrosis occurs in the kidneys. In some embodiments, the fibrosis is associated with focal segmental glomerulosclerosis (FSGS), glomerulonephritis, IgA nephropathy, diabetic nephropathy, transplant nephropathy, chronic allograft nephropathy, lupus nephritis, or unilateral ureteral obstruction-induced renal fibrosis. In some embodiments, the fibrosis is renal fibrosis.

In some embodiments, the fibrosis occurs in the lungs. In some embodiments, the fibrosis is associated with asthma, cystic fibrosis, chronic obstructive pulmonary disease (COPD), pulmonary arterial hypertension, acute respiratory distress syndrome (ARDS), or scleroderma lung disease. In some embodiments, the fibrosis is progressive massive fibrosis. In some embodiments, the fibrosis is pulmonary fibrosis (such as idiopathic pulmonary fibrosis). In some embodiments, the fibrosis is renal fibrosis characterized by tubulointerstitial fibrosis and glomerulosclerosis.

In some embodiments, the fibrosis occurs in the eyes. In some embodiments, the fibrosis is associated with age-related macular degeneration (AMD), glaucoma, diabetic macular edema, diabetic retinopathy, or dry eye disease.

In some embodiments, the fibrosis occurs in the heart. In some embodiments, the fibrosis is associated with heart failure, atherosclerosis, endomyocardial fibrosis, myocardial infarction, or atrial fibrosis. In some embodiments, the fibrosis is associated with congestive heart failure. In some embodiments, the fibrosis is cardiac fibrosis.

In some embodiments, the fibrosis occurs in soft tissue, bone marrow, skin, or peritoneum. In some embodiments, the fibrosis is mediastinal fibrosis, myelofibrosis (e.g., idiopathic- or drug-induced myelofibrosis), retroperitoneal fibrosis, nephrogenic systemic fibrosis, systemic sclerosis, or discoid lupus erythematosus. In some embodiments, the fibrosis occurs in the skin. In some embodiments, the fibrosis is associated with scleroderma, keloids, hypertrophic scarring, eosinophilic fasciitis, or dermatomyositis. In some embodiments, the fibrosis is skin scarring. In some embodiments, the compounds described herein are useful for reducing the severity of a scar. In some embodiments, the compounds described herein are useful for wound repair.

In some embodiments, the fibrosis occurs in a joint or joints. In some embodiments, the fibrosis occurs in the hands and/or fingers. In some embodiments, the fibrosis is athrofibrosis, Dupuytren's contracture, or adhesive capsulitis.

In some embodiments, the fibrosis occurs in the intestine. In some embodiments, the fibrosis is associated with Crohn's Disease.

In some embodiments, the fibrosis occurs in the penis. In some embodiments, the fibrosis is associated with Peyronie's disease.

In some embodiments, the fibrosis is the result of injury, surgery, or radiation. In some embodiments, the fibrosis is burn-induced. For example, in some embodiments, the fibrosis is burn-induced scarring and/or contraction. In some embodiments, the fibrosis is chemotherapy-induced (e.g., bleomycin-induced) pulmonary fibrosis. In some embodiments, the fibrosis is scarring following trabeculectomy in a patient with glaucoma. In some embodiments, the fibrosis is the result of an infection.

In some embodiments, the compounds described herein are useful for the treatment of idiopathic pulmonary fibrosis (“IPF”). In some embodiments, an individual in need of treatment has received a clinical and radiographic diagnosis of IPF. In some embodiments, an individual in need of treatment has undergone a surgical lung biopsy. In some embodiments, an individual in need of treatment has a percent predicted forced vital capacity (% FVC) greater than or equal to 50% at baseline. In some embodiments, an individual in need of treatment has a percent predicted diffusing capacity of the lungs for carbon monoxide (% DLCO) greater than or equal to 30% or 35%.

### IX. Interstitial Cystitis/Painful Bladder Syndrome

In some embodiments, the CB2 receptor-mediated disorder is interstitial cystitis. Interstitial cystitis (also known as painful bladder syndrome) is a chronic inflammatory condition of the bladder associated with urinary urgency, urinary frequency, and nocturia. CB2 receptors have been reported to be present in the bladder and its associated innervation, and CB2 receptors are upregulated in bladder after acute or chronic inflammation. CB2 receptors have therefore been suggested as a target for pharmacological treatment of bladder inflammation and associated pain. Neurosci Lett 445(1):130-134, 2008. Further, lipopolysaccharide (LPS)-induced bladder inflammation has been shown to increase expression of bladder CB2 (but not CB1) mRNA, and CB2 receptor agonist JWH015 has been shown to antagonize LPS-induced bladder inflammation (Tambaro et al., Eur J Pharmacol 2014). Accordingly, CB2 receptor agonists find use in the treatment of interstitial cystitis.

In some embodiments, an individual is diagnosed and/or assessed for a disease, condition, or disorder disclosed herein based on information from an imaging technique. For example, in some embodiments, an individual is diagnosed and/or assessed based on an ultrasound (e.g., FibroScan), CT (e.g., high resolution CT (HRCT)), or MRI scan. In some embodiments, an individual is diagnosed and/or assessed based on a pulmonary function test. For example, in some embodiments, a change in percent predicted forced volume vital capacity (FVC) from baseline to a defined endpoint is assessed. In some embodiments, an individual is diagnosed and/or assessed for pain based on the Western Ontario and McMasters Universities Osteoarthritis (WOMAC) Index.

Provided are compounds useful for the treatment of a CB2 receptor-mediated disorder. Also provided are methods for the treatment of a CB2 receptor-mediated disorder in an individual in need thereof, comprising administering to the individual a therapeutically effective amount of a compound described herein. Also provided are compounds useful for the treatment of pain. Also provided are compounds useful for the treatment of osteoarthritis. Also provided are compounds useful for the treatment of a liver disease selected from liver fibrosis, primary biliary cirrhosis, and nonalcoholic steatohepatitis. In some embodiments, the liver disease is liver fibrosis. In some embodiments, the liver disease is primary biliary cirrhosis. In some embodiments, the liver disease is nonalcoholic steatohepatitis. Also provided are compounds useful for the treatment of bone and joint pain. Also provided are compounds useful for the treatment of bone pain. Also provided are compounds useful for the treatment of joint pain. Also provided are compounds useful for the treatment of pain associated with osteoarthritis. Also provided are compounds useful for the treatment of osteoporosis. Also provided are compounds useful for the treatment of hyperalgesia. Also provided are compounds useful for the treatment of allodynia. Also provided are compounds useful for the treatment of inflammatory pain. Also provided are compounds useful for the treatment of inflammatory hyperalgesia. Also provided are compounds useful for the treatment of neuropathic pain. Also provided are compounds useful for the treatment of neuropathic hyperalgesia. Also provided are compounds useful for the treatment of acute nociception. Also provided are compounds useful for the treatment of muscle pain. Also provided are compounds useful for the treatment of dental pain. Also provided are compounds useful for the treatment of migraine and other headache pain. Also provided are compounds useful for the treatment of pain that occurs as an adverse effect of therapeutics. Also provided are compounds useful for the treatment of pain associated with a disorder selected from: cancer, multiple sclerosis, allergic reactions, nephritic syndrome, scleroderma, thyroiditis, diabetic neuropathy, fibromyalgia, HIV related-neuropathy, sciatica, and autoimmune conditions. Also provided are compounds useful for the treatment of multiple sclerosis-associated spasticity. Also provided are compounds useful for the treatment of autoimmune disorders. Also provided are compounds useful for the treatment of an autoimmune disorder selected from the group consisting of: multiple sclerosis, Guillan-Barré syndrome, polyradiculoneuropathy, chronic inflammatory demyelination, rheumatoid arthritis, psoriatic arthritis, ankylosing spondylarthritis, and reactive arthritis. Also provided are compounds useful for the treatment of allergic reactions. Also provided are compounds useful for the treatment of an allergic reaction associated with a disorder selected from: atopic dermatitis, pruritis, urticaria, asthma, conjunctivitis, allergic rhinitis, and anaphylaxis. Also provided are compounds useful for the treatment of CNS inflammation. Also provided are compounds useful for the treatment of CNS inflammation associated with a disorder selected from: Alzheimer's disease, stroke, dementia, amyotrophic lateral sclerosis, and human immunodeficiency virus. Also provided are compounds useful for the treatment of atherosclerosis. Also provided are compounds useful for the treatment of undesired immune cell activity and inflammation associated with a disorder selected from: osteoarthritis, anaphylaxis, Behcet's disease, graft rejection, vasculitis, gout, spondylitis, viral disease, bacterial disease, lupus, inflammatory bowel disease, autoimmune hepatitis, and type 1 diabetes mellitus. Also provided are compounds useful for the treatment of age-related macular degeneration. Also provided are compounds useful for the treatment of cough. Also provided are compounds useful for the treatment of leukemia. Also provided are compounds useful for the treatment of lymphoma. Also provided are compounds useful for the treatment of CNS tumors. Also provided are compounds useful for the treatment of prostate cancer. Also provided are compounds useful for the treatment of Alzheimer's disease. Also provided are compounds useful for the treatment of stroke-induced damage. Also provided are compounds useful for the treatment of dementia. Also provided are compounds useful for the treatment of amyotrophic lateral sclerosis. Also provided are compounds useful for the treatment of Parkinson's disease.

In some embodiments, the disorder is a CB2 receptor-mediated disorder. In some embodiments, the CB2 receptor-mediated disorder is pain or a condition related thereto. In some embodiments, the CB2 receptor-mediated disorder is osteoarthritis. In some embodiments, the CB2 receptor-mediated disorder is fibrosis or a condition related thereto. In some embodiments, the CB2 receptor-mediated disorder is liver fibrosis. In some embodiments, the CB2 receptor-mediated disorder is primary biliary cirrhosis. In some embodiments, the CB2 receptor-mediated disorder is nonalcoholic steatohepatitis. In some embodiments, the CB2 receptor-mediated disorder is diabetic neuropathy. In some embodiments, the CB2 receptor-mediated disorder is interstitial cystitis. In some embodiments, the CB2 receptor-mediated disorder is pain associated with interstitial cystitis. In some embodiments, the CB2 receptor-mediated disorder is endometriosis. In some embodiments, the CB2 receptor-mediated disorder is pain associated with endometriosis.

### Compositions and Formulations

The compounds described herein can be administrated in a wide variety of oral and parenteral dosage forms. One of skill in the art will understand that the dosage forms may comprise, as the active component, either a compound described herein or a pharmaceutically acceptable salt, hydrate, or solvate of a compound described herein.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound described herein in a suitable liquid vehicle and filter sterilizing the solution before filling and sealing an appropriate vial or ampule. These are just a few examples of the many appropriate methods well known in the art for preparing dosage forms.

A compound described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20th Edition, 2000, Lippincott Williams & Wilkins, (editors: Gennaro et al.).

While it is possible that a compound described herein may be administered as a raw or pure chemical, it is preferable to present the compound or active ingredient as a pharmaceutical formulation or as a composition further comprising a pharmaceutically acceptable carrier.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation, or transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive, and a removable protective layer with a release liner. One of skill in the art will understand and appreciate the techniques appropriate for manufacturing a desired efficacious transdermal patch based upon the needs of one of skill in the art.

The compounds described herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds described herein or a solvate, hydrate or physiologically functional derivative thereof can be used as active ingredients in pharmaceutical compositions, specifically as CB2 receptor agonists. The term “active ingredient,” defined in the context of a “pharmaceutical composition,” refers to a component of a pharmaceutical composition that provides the primary pharmacological effect, as opposed to an “inactive ingredient” which would generally be recognized as providing no pharmaceutical benefit.

For preparing pharmaceutical compositions from the compounds described herein, the selection of a suitable pharmaceutically acceptable carrier can be either solid, liquid, or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, one of skill in the art would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethyl cellulose, a low melting wax, cocoa butter and the like. The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams, or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds described herein may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethyl cellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds described herein may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds described herein or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds described herein as an aerosol can be prepared by processes well known to the person skilled in the art. For their preparation, for example, solutions or dispersions of the compounds described herein in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds described herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain compounds described herein which contain a carboxylic acid functional group may optionally exist as pharmaceutically acceptable salts containing non-toxic, pharmaceutically acceptable metal cations and cations derived from organic bases. Representative metals include, but are not limited to, aluminum, calcium, lithium, magnesium, potassium, sodium, zinc and the like. In some embodiments the pharmaceutically acceptable metal is sodium. Representative organic bases include, but are not limited to, benzathine (N1,N2-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et al., J Pharmaceutical Sciences, 66:1-19 (1977).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds described herein may form solvates with standard low molecular weight solvents using methods known to one of skill in the art.

Compounds described herein can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds described herein containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems vol. 14 of the A.C.S. Symposium Series; and in Bioreversible Carriers in Drug Design, ed. Edward B. Roche, American Pharmaceutical Association and Pergamon Press, 1987.

Some embodiments include a method of producing a pharmaceutical composition for combination therapy comprising admixing at least one compound according to any of the compound embodiments disclosed herein, together with at least one known pharmaceutical agent as described herein and a pharmaceutically acceptable carrier.

It will be apparent to those skilled in the art that the dosage forms described herein may comprise, as the active component, either a compound described herein, a pharmaceutically acceptable salt of a compound described herein, a solvate or hydrate of a compound described herein, or a solvate or hydrate of a pharmaceutically acceptable salt of a compound described herein. Moreover, various hydrates and solvates of the compounds described herein and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, vol. 95, Marcel Dekker, Inc., New York, 1999. Accordingly, one aspect of the present disclosure pertains to methods of administering hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

The present disclosure includes all isotopes of atoms occurring in salts and crystalline forms thereof. Isotopes include those atoms having the same atomic number but different mass numbers. One aspect of the present invention includes every combination of one or more atoms in the present salts and crystalline forms thereof that is replaced with an atom having the same atomic number but a different mass number. One such example is the replacement of an atom that is the most naturally abundant isotope, such as ¹H or ¹²C, found in one the present salts and crystalline forms thereof, with a different atom that is not the most naturally abundant isotope, such as ²H or ³H (replacing ¹H), or ¹¹C, ¹³C, or ¹⁴C (replacing ¹²C). A salt wherein such a replacement has taken place is commonly referred to as being isotopically-labeled. Isotopic-labeling of the present salts and crystalline forms thereof can be accomplished using any one of a variety of different synthetic methods known to those of skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as those prepared during synthesis, preformulation, and the like, and pharmaceutical compositions, such as those prepared with the intent of using in a mammal for the treatment of one or more of the disorders described herein, comprising one or more of the present salts and crystalline forms thereof, wherein the naturally occurring distribution of the isotopes in the composition is perturbed. Another aspect of the present invention includes compositions and pharmaceutical compositions comprising salts and crystalline forms thereof as described herein wherein the salt is enriched at one or more positions with an isotope other than the most naturally abundant isotope. Methods are readily available to measure such isotope perturbations or enrichments, such as mass spectrometry, and for isotopes that are radio-isotopes additional methods are available, such as radio-detectors used in connection with HPLC or GC.

Doses and Dosage Regimens

The dose when using the compounds described herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds described herein. Representative doses include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the individual and will ultimately be at the discretion of the attendant physician or clinician. In general, one of skill in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the individual, the severity of the disease, the route of administration, pharmacological considerations such as the activity, efficacy, pharmacokinetic and toxicology profiles of the particular compound employed, whether a drug delivery system is utilized, whether an acute or chronic disease state is being treated or prophylaxis conducted or whether further active compounds are administered in addition to the compounds described herein such as part of a drug combination. The dosage regimen for treating a disease condition with the compounds and/or compositions described herein is selected in accordance with a variety factors as cited herein. Thus, the actual dosage regimen employed may vary widely and therefore may deviate from a preferred dosage regimen and one of skill in the art will recognize that dosage and dosage regimen outside these typical ranges can be tested and, where appropriate, may be used in the methods described herein.

In some embodiments, the dose of a compound or pharmaceutically acceptable salt, solvate, or hydrate thereof is about 5 mg, about 10 mg, about 20 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 325 mg, about 350 mg, about 375 mg, about 400 mg, about 425 mg, about 450 mg, about 475 mg, or about 500 mg. In some embodiments, the dose is a daily dose. In some embodiments, the dose is a twice daily dose. In some embodiments, the dose is a three times daily dose. In some embodiments, the dose is a four times daily dose. In some embodiments, the dose is titrated.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

Administration

In some embodiments, the individual in need of treatment is an adult. In some embodiments, the individual in need of treatment is an adolescent. In some embodiments, the individual in need of treatment is a child. In some embodiments, the individual in need of treatment is aged 13-17 years. In some embodiments, the individual in need of treatment is aged 10-17 years. In some embodiments, the individual in need of treatment is aged 5-16 years.

It is noted that when the CB2 receptor agonists are utilized as active ingredients in pharmaceutical compositions, these are not necessarily intended for use in humans only, but may be used for non-human mammals as well. Recent advances in the area of animal health-care mandate that consideration be given for the use of active agents, such as CB2 receptor agonists, for the treatment of a CB2 receptor-associated disease or disorder in companionship animals (e.g., cats, dogs, etc.) and in livestock animals (e.g., horses, cows, etc.) Those of ordinary skill in the art are readily credited with understanding the utility of such compounds in such settings.

In some embodiments, the compounds described herein are for use in acute treatment. In some embodiments, the compounds described herein are for use in short-term treatment. In some embodiments, the compounds described herein are for use in chronic treatment. In some embodiments, the compounds described herein are for use in long-term treatment. In some embodiments, the compounds described herein are for use in maintenance treatment. In some embodiments, the duration of treatment is selected from at least about: 1 week, 2 weeks, 3, weeks, 4 weeks, 5 weeks, 6 weeks, 7 weeks, 8 weeks, 9 weeks, 12 weeks, 6 months, 9 months, 1 year, 18 months, 2 years, 3 years, 4 years, and 5 years.

In some embodiments, the compounds described herein are useful for slowing the progression of a disorder. In some embodiments, the compounds described herein are useful for maintaining the stage and/or severity of a disorder. In some embodiments, the compounds described herein are useful for maintaining the stage and/or severity of a disorder for at least about: 12 weeks, 6 months, 9 months, 1 year, 18 months, 2 years, 3 years, 4 years, and 5 years.

In some embodiments, the methods described herein further comprise the step of providing an individual with biochemical feedback; acupuncture; hypnosis; behavioral intervention; support services; and/or psychosocial treatment.

In some embodiments, the compounds described herein are for use in monotherapy. In some embodiments, the compounds described herein are for use in combination therapy. In some embodiments, the compounds described herein are for use as an adjunct therapy. In some embodiments, the compounds described herein are for use in combination with an analgesic. In some embodiments, the compounds described herein are for use in combination with an antidiabetic agent. In some embodiments, the compounds described herein are for use in combination with an osteoarthritis agent. In some embodiments, the compounds described herein are for use in combination with an anticancer agent. In some embodiments, the compounds described herein are for use in combination with an inhibitor of inherent multidrug resistance, an anti-emetic agent, an agent useful in the treatment of anemia, an agent useful in the treatment of neutropenia, an immunologic-enhancing agent, or an anticancer agent. In some embodiments, the compounds described herein are for use in combination with an anti-inflammatory agent. In some embodiments, the compounds described herein are for use in combination with an anti-coagulation agent. In some embodiments, the compounds described herein are for use in combination with a corticosteroid. In some embodiments, the compounds described herein are for use as an adjunct to pirfenidone. In some embodiments, the compounds described herein are for use as an adjunct to nintedanib. In some embodiments, the compounds described herein are for use in combination with pirfenidone, nintedanib, and/or inhaled N-acetylcysteine (NAC) for the treatment or prevention of idiopathic pulmonary fibrosis.

In some embodiments, a health care provider orders, authorizes, or recommends the use of a compound or pharmaceutical composition. In some embodiments, a CB2 receptor agonist is prescribed to an individual in need thereof. In some embodiments, a health care provider orally advises, recommends, or authorizes the use of a compound, dosage regimen, or other treatment to an individual. The health care provider may or may not provide a written prescription for the compound, dosage regimen, or treatment. Further, the health care provider may or may not provide the compound or treatment to the individual. For example, the health care provider can advise the individual where to obtain the compound without providing the compound. In some embodiments, a health care provider can provide a written prescription for the compound, dosage regimen, or treatment to the individual. A prescription can be written on paper or recorded on electronic media. In addition, a prescription can be called in (oral) or faxed in (written) to a pharmacy or a dispensary. In some embodiments, a sample of the compound or treatment is given to the individual. As used herein, giving a sample of a compound constitutes an implicit prescription for the compound. Different health care systems around the world use different methods for prescribing and administering compounds or treatments, and these methods are encompassed by the disclosure herein. A health care provider can include, for example, a physician, nurse, nurse practitioner, or other health care professional who can prescribe or administer compounds (drugs) for the disorders described herein. In addition, a health care provider can include anyone who can recommend, prescribe, administer, or prevent an individual from receiving a compound or drug, including, for example, an insurance provider.

In some embodiments, a health care provider directly provides a compound to an individual in the form of a sample, or directly provides a compound to an individual by providing an oral or written prescription for the compound. In some embodiments, an individual obtains a compound by themself without the involvement of a health care provider. In some embodiments, the individual internalizes the compound.

The compounds described herein or pharmaceutically acceptable salts, solvates, or hydrates thereof may be administered sequentially or concurrently with the one or more other supplemental agents identified herein. The amounts of formulation and pharmacologic agent depend, for example, on what type of pharmacologic agent(s) are used, and the scheduling and routes of administration. Supplemental agent delivery may be via any suitable method known in the art including orally, inhalation, injection, etc.

In Vitro and In Vivo Assays

As one of skill in the art will recognize, receptor internalization can be measured using a number of methods, including but not limited to measuring a loss of labeled receptor from the cell surface (e.g., using flow cytometry) measuring the appearance of receptors internalized in the cell (e.g., in characteristic punctate intracellular vesicles), and/or measuring the return of receptors recycled to the cell surface. For example, the number, density, and/or staining intensity of granules in the cell can be quantified. Receptor internalization can be measured using any appropriate method known to those of skill in the art. In some embodiments, receptor internalization is measured as the loss of receptors from the cell surface. In some embodiments, receptor internalization is measured as the appearance of receptors inside the cell. For example, in some embodiments, receptor internalization is measured as the appearance of internalized receptors in intracellular vesicles. In some embodiments, receptor internalization is measured using epitope-tagged receptors. In some embodiments, receptor internalization is measured using antibody-labeled receptors. In some embodiments, receptor internalization is measured using fluorescently labeled receptors. For example, in some embodiments, receptor internalization is measured as a change in fluorescence intensity at the cell surface and/or inside the cell. In some embodiments, the number, density, and/or staining intensity of fluorescent granules in the cell are quantified. In some embodiments, receptor internalization is measured using an immunoassay. In some embodiments, receptor internalization is measured using a Western blot. In some embodiments, receptor internalization is measured using immunofluorescence. In some embodiments, receptor internalization is measured using fluorescence microscopy. In some embodiments, receptor internalization is measured using a flow cytometry assay. In some embodiments, receptor internalization is measured using enzyme complementation. In some embodiments, receptor internalization is quantified using high content analysis.

In some embodiments, internalization of the CB2 receptor is measured. In some embodiments, the receptor internalization level is measured as internalization efficacy relative to another CB2 receptor agonist. In some embodiments, the receptor internalization level is about, or at least about, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% the internalization level for a full CB2 receptor internalization agonist. As used herein, the term “full CB2 receptor internalization agonist” refers to a compound with an internalization efficacy that is about, or at least about, equal to that of CP55,940, Compound 699, Compound 841, Compound 919, and/or Compound 765. In some embodiments, the receptor internalization level is measured relative to CP55,940. In some embodiments, the receptor internalization level is measured relative to Compound 699. In some embodiments, the receptor internalization level is measured relative to a CB2 receptor agonist with an internalization level less than CP55,940 and/or Compound 699. In some embodiments, the receptor internalization level is measured relative to a CB2 receptor agonist with an internalization level comparable to CP55,940 and/or Compound 699. In some embodiments, the receptor internalization level is measured relative to a CB2 receptor agonist with an internalization level about equal to CP55,940 and/or Compound 699. In some embodiments, the receptor internalization level is measured relative to a CB2 receptor agonist with an internalization level greater than CP55,940 and/or Compound 699. In some embodiments, the receptor internalization level is about, or at least about, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% the internalization level for CP55,940. In some embodiments, the receptor internalization level is about, or at least about, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% the internalization level for Compound 699.

In some embodiments, the EC50 for receptor internalization following contact with a compound described herein is less than, or less than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 47, 49, or 50 nM.

In some embodiments, agonism of a compound for the CB2 receptor is measured. In some embodiments, agonism is determined by the in vitro potency of a compound. In some embodiments, agonism is measured using a second messenger assay. In some embodiments, agonism is measured using a cAMP assay. In some embodiments, agonism is measured using a β-arrestin assay. In some embodiments, agonism is measured using a GTP-γS binding assay. In some embodiments, agonism is measured using a reporter gene assay. In some embodiments, agonism is measured using a biomarker. In some embodiments, agonism is quantified as EC50.

In some embodiments, binding affinity of a compound is measured. In some embodiments, binding affinity is quantified as Ki. In some embodiments, competitive binding is measured. In some embodiments, displacement of a bound compound is measured. For example, in some embodiments, the displacement of CP55,940 is determined.

In some embodiments, the IC50 for a compound described herein in a β-arrestin assay is less than, or less than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 47, 49, or 50 nM.

In some embodiments, selectivity of a compound for the CB2 receptor is measured. In some embodiments, selectivity refers to the relative in vitro potency of a compound for the CB2 receptor and another receptor. For example, in some embodiments, selectivity refers to the relative in vitro potency of a compound for the CB2 receptor versus the CB1 receptor. In some embodiments, in vitro potency is measured using a second messenger assay. In some embodiments, in vitro potency is measured using a cAMP assay. In some embodiments, selectivity is determined by comparing data generated using a β-arrestin assay. In some embodiments, selectivity is determined by comparing data generated from a GTP-γS binding assay. In some embodiments, selectivity is determined by comparing data generated from a reporter gene assay. In some embodiments, selectivity is determined by comparing data generated for a biomarker. In some embodiments, in vitro potency is quantified as EC50. In some embodiments, selectivity refers to the relative binding affinity of an agonist for the CB2 receptor and another receptor. In some embodiments, binding affinity is quantified as Ki.

In some embodiments, selectivity is assessed for the mouse, rat, or human CB2 receptor. In some embodiments, selectivity is assessed for the human CB2 receptor. In some embodiments, selectivity is assessed for the CB2 receptor versus the CB1 receptor. In some embodiments, selectivity is assessed for the human CB2 receptor versus the human CB1 receptor. In some embodiments, a compound described herein exhibits about, or at least about, 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1750-fold, 2000-fold, 2500-fold, 3000-fold, 3500-fold, 4000-fold, 4500-fold, 5000-fold, 6000-fold, 7000-fold, 8000-fold, 9000-fold, or 10000-fold selectivity for the CB2 receptor versus the CB1 receptor. In some embodiments, a compound described herein exhibits about, or at least about, 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1750-fold, 2000-fold, 2500-fold, 3000-fold, 3500-fold, 4000-fold, 4500-fold, 5000-fold, 6000-fold, 7000-fold, 8000-fold, 9000-fold, or 10000-fold selectivity for the human CB2 receptor versus the human CB1 receptor.

In some embodiments, in vivo efficacy of a compound is measured. In some embodiments, in vivo efficacy is measured for a disorder described herein. In some embodiments, in vivo efficacy is measured for pain. In some embodiments, in vivo efficacy is measured for fibrosis. In some embodiments, in vivo efficacy is measured using diagnostic criteria described herein. In some embodiments, in vivo efficacy is measured about, or at least about, 0.5, 1, 1.5, 2, 2.5, 3, 3.5, 4, 4.5, 5, 5.5, 6, 6.5, 7, 7.5, or 8 hours following dosing with a compound described herein. In some embodiments, in vivo efficacy is measured in an animal model. In some embodiments, in vivo efficacy is measured in a non-human mammal. In some embodiments, in vivo efficacy is measured in a human. In some embodiments, in vivo efficacy is measured in an animal model. In some embodiments, the animal model is a model for a CB2 receptor-mediated disorder. In some embodiments, the animal model is a model for pain or conditions related thereto. In some embodiments, the animal model is a model for fibrosis or conditions related thereto. In some embodiments, the animal model is a Freund's complete adjuvant (FCA)-induced hyperalgesia model. In some embodiments, the animal model is a capsaicin-induced model of hyperalgesia and/or allodynia. In some embodiments, the animal model is a Zucker diabetic fatty (ZDF) rat. In some embodiments, the animal model is a streptozotocin (STZ)-treated rat. In some embodiments, the animal model is a model of neuropathic pain, such as a chronic constriction injury model of neuropathic pain. In some embodiments, the animal model is a bile duct ligation model. In some embodiments, the animal model is a hepatic fibrosis model. In some embodiments, the animal model is a NASH model. In some embodiments, the animal model is a pulmonary fibrosis model, such as a bleomycin-induced pulmonary fibrosis model. In some embodiments, the animal model is a dermal fibrosis model. In some embodiments, the animal model is a model of acute injury, such as acute kidney injury. In some embodiments, the animal model is a cholestatic liver injury model. In some embodiments, the animal model is an experimental autoimmune encephalomyelitis (EAE) model. In some embodiments, the animal model is an occlusion model of stroke. In some embodiments, the animal model is a model of atherosclerosis. In some embodiments, the animal model is a cyclophosphamide (CYP)-induced cystitis model.

Each embodiment described herein is to be applied mutatis mutandis to each and every other embodiment unless specifically stated otherwise.

One of skill in the art will recognize that the methods of treatment and/or methods of administration disclosed herein can be modified to recite Swiss-type use claims, second medical use claims, or any other appropriate claim type for a given jurisdiction.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing or administering a compound described herein can be separated into two methods—one reciting prescribing a compound described herein and the other reciting administering a compound described herein. In addition, for example, a method that recites prescribing a compound described herein and a separate method reciting administering a compound described herein can be combined into a single method reciting prescribing and/or administering a compound described herein.

As will be recognized, the steps of the methods of the present invention need not be performed any particular number of times or in any particular sequence. Additional objects, advantages, and novel features of this invention (including other uses of the compounds) will become apparent to those skilled in the art upon examination of a review of the disclosure and the following examples thereof, which are intended to be illustrative and not intended to be limiting.

Those skilled in the art will appreciate that the invention(s) described herein is susceptible to variations and modifications other than those specifically described, including but not limited to functionally equivalent compounds, pharmaceutical compositions, and methods. It is to be understood that the invention(s) includes all such variations and modifications. The invention(s) also includes all of the steps, features, compositions, and compounds referred to or indicated in this specification, individually or collectively, and any and all combinations or any two or more of said steps or features unless specifically stated otherwise.

### EXAMPLES

### Example 1—Radioligand Binding Assay

CHO cells stably expressing a full length human CB2 receptor (NCBI Reference Sequence NP_001832.1 with and without a Q63R substitution) were collected, washed in ice cold PBS, and centrifuged at 48,000×g for 20 minutes at 4° C. The cell pellet was then collected, resuspended in wash buffer (20 mM HEPES, pH 7.4 and 1 mM EDTA), homogenized on ice using a Brinkman Polytron, and centrifuged at 48,000×g for 20 minutes at 4° C. The resultant pellet was resuspended in ice cold 20 mM HEPES, pH 7.4, homogenized again on ice, recentrifuged for 20 minutes at 4° C., and membrane pellets were then stored at −80° C. until needed.

Radioligand binding assays for human CB2 receptors were performed using two different agonist radioligands, [³H]CP55,940 and [³H]WIN55,212-2 under similar assay conditions. For both assays, nonspecific binding was determined in the presence of 10 μM of unlabeled compound. Competition experiments entailed the addition of 20 μL of assay buffer (50 mM Tris, pH 7.4, 2.5 mM EDTA, 5 mM MgCl₂, and 0.5 mg/mL of fatty acid free BSA) containing test compound (concentrations ranging from 1 pM to 100 μM), 25 μL of radioligand (1 nM final assay concentration for [³H]CP55,904 and [³H]WIN55, 212-2), and 50 μL of membranes (20 μg/mL final protein for both assays). Incubations were conducted for 1 hour at room temperature, and assay plates were filtered under reduced pressure over GF/B filters, washed with assay buffer, and dried overnight in a 50° C. oven. 25 μL of BetaScint scintillation cocktail was then added to each well, and plates were read in a Packard TopCount (Perkin Elmer, Waltham, Mass.) scintillation counter.

To determine selectivity, competition curves were fit to a nonlinear least squares curve fitting program to obtain IC₅₀ values for the radioligand biding data. Ki values were determined from IC₅₀ values using the Cheng-Prusoff equation and the Kd value for each radioligand-receptor pair. Mean Ki values were calculated from the mean of the log Ki values. Selectivity for the CB2 receptor versus the CB1 receptor was obtained by dividing the CB2 receptor Ki value by the CB1 receptor Ki value.

### Example 2: cAMP Assay

HTRF® cAMP assays for the CB2 receptor were performed according to the manufacturer instructions (cAMP Dynamic 2 Assay Kit; Catalog #62AM4PEJ, Cisbio Bioassays, Bedford, Mass.). CHO-K1 cells stably expressing recombinant CB2 receptor were harvested and suspended in assay buffer (PBS containing 0.5 mM IBMX, 2 μM forskolin, and 0.1% fatty acid free BSA) at a density of 300,000 cells per mL. The cell suspension was dispensed into 384-well assay plates (Proxiplate, Catalog #6008280, PerkinElmer, Fremont, Calif.) at 5 μL per well along with a cAMP standard curve. Test compounds were dissolved in DMSO, serially diluted in DMSO, and then further diluted in assay buffer to achieve 2× concentrations in 1% DMSO. The diluted compounds were then transferred to the assay plates (5 μL per well). Following incubation at room temperature for one hour, 5 μL of cAMP-D2 reagent diluted in lysis buffer was added to each well followed by 5 μL of cryptate reagent. Plates were then incubated at room temperature for one hour prior to reading. Time resolved fluorescence measurements were collected on an appropriate HTRF®-capable microplate reader (e.g., EnVision™ (Perkin Elmer, Waltham, Mass.) or PHERAStar (BMG Labtech Inc., Durham, N.C.)).

Test compound counts from the microplate reader were fit to the cAMP standard curve on each plate to calculate the concentration of cAMP in each well. Dose response curves were generated using a nonlinear least squares curve fitting program to obtain EC50 values. Dose-response experiments were generally performed with 8 or 10 serial dilutions of test compound with triplicate determinations for each concentration. Assay performance was monitored by inclusion of a standard CB2 reference agonist (such as CP55,940) on each assay plate and agonist efficacies were reported relative to that of the reference agonist.

### Example 3—β-Arrestin Assay

β-arrestin recruitment assays were performed using the PathHunter® arrestin assay system from DiscoveRx (Fremont, Calif.). The assays were performed in CB2-receptor-expressing stable cell lines generated in the PathHunter® CHO-K1 parental cell line.

β-Arrestin recruitment assays were performed according to manufacturer instructions. Briefly, PathHunter CHO-K1 cells stably expressing the recombinant human CB2 receptor were seeded into 384-well microtiter assay plates (OptiPlate 384-Plus, Catalog #6007299, Perkin Elmer, Waltham, Mass.) at a density of 5,000 cells per well in 20 μL serum-free growth medium (e.g., OptiMEM, Corning Inc., Corning, N.Y.) and cultured in a humidified 37° C. incubator overnight. Plates were then removed from the incubator and allowed to equilibrate to room temperature for one hour. Test compounds dissolved in DMSO were serially diluted in DMSO and then further diluted in OptiMEM to achieve 5× concentrations in 2.5% DMSO. Aliquots (5 μL) of diluted test compounds were added to assay plates, which were then incubated at room temperature for two hours. Lysis/detection reagents (12 μL total) were then added and the plates were sealed and incubated for an additional two hours at room temperature. Plates were then read on an appropriate plate reader (e.g., EnVision™ (Perkin Elmer, Waltham, Mass.) or PHERAStar (BMG Labtech Inc., Durham, N.C.)). Dose-response curves were generated using a minimum of eight different test concentrations and triplicate determinations were made at each test concentration. Assay performance was monitored by inclusion of a standard CB2 reference agonist (such as CP55,940) on each assay plate and agonist efficacies were reported relative to that of the reference agonist.

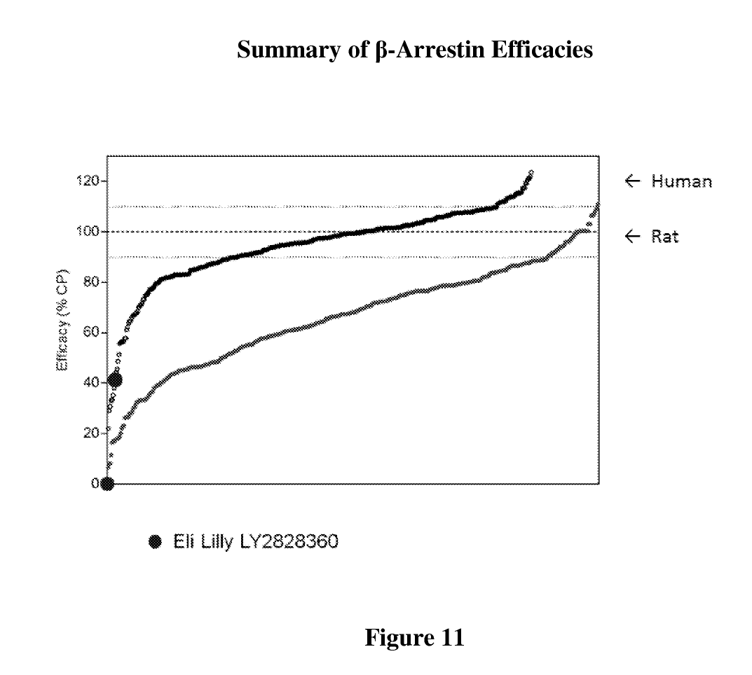

A summary of the efficacies of CB2 receptor agonists with potencies less than 500 nM in β-arrestin assays is provided in FIG. 11, including Eli Lilly Compound LY2828360 as an example.

### Example 4—Receptor Internalization Assay (High Content Analysis)

Receptor internalization assays for the CB2 receptor were performed using a high content imaging system (e.g., IN Cell Analyzer, GE Healthcare Life Sciences, Pittsburgh, Pa.), or by conventional fluorescence microscopy with accessory image analysis software.

Briefly, CHO-K1 cells stably expressing full length recombinant human CB2 receptors (NCBI Reference Sequence NP_001832.1) with and without a Q63R substitution fused with an N-terminal hemagglutinin (HA) tag were seeded into poly-d-lysine coated 96-well view plates (Catalog #6005182, Perkin Elmer, Waltham, Mass.) at a density of 6,000 per well in 100 μL of serum-free growth medium (RPMI Media, Gibco) and cultured in a humidified 37° C. incubator overnight. On the day of the assay, growth media was removed from the cell plate and replaced with 25 μL pre-warmed RPMI media. Additionally, 25 μL of pre-warmed RPMI media containing a fluorescent nuclear stain (Hoescht 33342 dye (1:500 dilution); Catalog #H-1399, Invitrogen,) and fluorophore-labeled antibody (Alexa-488 anti-HA monoclonal antibody (1:50 dilution); Catalog #A-21287, Invitrogen, Waltham, Mass.) was added to the cell plate. Test compounds or CP55,940 dissolved in DMSO were serially diluted in DMSO and then further diluted in RPMI to achieve 3× concentrations in 0.5% DMSO. Aliquots (25 μL) of diluted test compounds were added to assay plates, which were then incubated in a water bath at 37° C. for 1 hour. Compound containing media was removed at the end of the treatment period by inversion of the plate. The assay plate was washed twice with PBS (150 μL/well) and the cells were then fixed with 2% formaldehyde/PBS solution for 10 minutes at room temperature. Fluorescence microscopy images were captured for each well on the plate (minimum of 6 per well). The internalization of anti-HA antibody-labeled CB2 receptors results in redistribution of fluorescence from the cell surface to intracellular endosomes, which appear as punctate fluorescent granules within the cell. Quantification of receptor internalization was performed by measuring the Alexa-488 anti-HA monoclonal antibody-stained intracellular granule fluorescence intensities normalized per cell number in each well. Dose-response curves were generated using a minimum of 8 different test concentrations and triplicate determinations were made at each test concentration. Assay performance was monitored by inclusion of a standard CB2 reference agonist such as CP55,940 and agonist internalization efficacies were reported relative to that of the reference agonist.

### Example 5—Receptor Internalization Assay (Enzyme Complementation)

Receptor internalization assays for the CB2 receptor were performed using the PathHunter® Total GPCR Internalization system from DiscoverRx (Fremont, Calif.). The assay was performed in CB2-receptor-expressing stable cell lines generated in the PathHunter® U2OS parental cell line.

Total GPCR internalization assays were performed per the manufacturer's instructions. Briefly, PathHunter® U2OS cells stably expressing the recombinant human or rat CB2 receptor were seeded into 96-well microtiter assay plates (Catalog #3610, Corning Inc., Corning, N.Y.) at a density of 20,000 cells per well in 100 μL growth medium and cultured in a humidified 37° C. incubator overnight. Growth medium was exchanged the following day for 100 μL serum-free medium (OptiMEM), and cells were incubated overnight in a humidified 37° C. incubator. On the day of the assay, test compounds dissolved in DMSO were serially diluted in DMSO and then further diluted in OptiMEM to achieve 11× concentrations in 5.5% DMSO. Aliquots (10 μL) of diluted test compounds were added to the assay plates, which were then incubated in a humidified 37° C. incubator for 3 hours. Lysis/detection reagents (55 μL total) were then added and the plates were incubated for an additional 1 hour at room temperature. Plates were then read on an appropriate plate reader (e.g., EnVision (Perkin Elmer, Waltham, Mass.) or PheraStar (BMG)). Dose-response curves were generated using a minimum of 8 different test concentrations and triplicate determinations were made at each test concentration. Assay performance was monitored by inclusion of a standard CB2 reference agonist such as CP55,940 and agonist internalization efficacies were reported relative to that of the reference agonist.

### Example 6—CB2 Receptor Selectivity

Competition curves were fit to a nonlinear least squares curve fitting program to obtain EC₅₀ values for the β-arrestin data. Selectivity for the CB2 receptor versus the CB1 receptor was obtained by dividing the CB2 receptor EC₅₀ value by the CB1 receptor EC₅₀ value.

### Example 7—Osteoarthritis Pain Model

Injection of monosodium iodoacetate (MIA) into a joint (J Rheumatol 14:130-1, 1987) inhibits the activity of glyceraldehyde-3-phosphate dehydrogenase in chondrocytes, resulting in disruption of glycolysis and eventually in cell death. The progressive loss of chondrocytes results in histological and morphological changes of the articular cartilage, closely resembling those seen in osteoarthritis patients.

Osteoarthritis was induced in 200 g male Sprague Dawley rats. After brief anesthesia by isoflurane, rats received a single intra-articular injection of MIA (2 mg) (Catalog #19148, Sigma Aldrich, Saint Louis, Mo.) dissolved in 0.9% sterile saline in a 50 μL volume administered through the patella ligament into the joint space of the left knee with a 30 G needle. Following injection, animals were allowed to recover from anesthesia before being returned to the main housing vivarium.

Typically during disease progression, there is an inflammation period of 0-7 days post-intra-articular injection followed by progressive degeneration of the cartilage and subchondral bone from days 14-55. Efficacy studies with a compound described herein took place from day 14 onwards and were performed twice a week with at least three days of wash-out in between each assay. Three different assays can be used to measure pain. Tactile allodynia can be measured via von Frey assay, hind limb paw weight distribution can be monitored using an incapacitance tester (Columbus Instruments, Columbus, Ohio), and hind limb grip strength can be measured using a grip strength meter (Columbus Instruments, Columbus, Ohio). Briefly, the von Frey assay was performed using the standard up/down method with von Frey filaments. Hind paw weight distribution was determined by placing rats in a chamber so that each hind paw rested on a separate force plate of the incapacitance tester. The force exerted by each hind limb (measured in grams) was averaged over a three second period. Three measurements are taken for each rat, and the change in hind paw weight distribution was calculated. Peak hind limb grip force was conducted by recoding the maximum compressive force exerted on the hind limb mesh gauge set on the grip strength meter. During testing, each rat was restrained and the paw of the injected knee was allowed to grip the mesh. The animal was then pulled in an upward motion until their grip was broken. Each rat was tested three times, with the contralateral paw used as a control.

A baseline was established for animals prior to administration of compounds. The MIA-treated groups of rats were then dosed with either vehicle (PEG400, orally), test compound (at 3 mg/kg, 10 mg/kg, and 30 mg/kg, orally), or morphine (3 mg/kg, subcutaneously). The dosing volume was 500 μL. One hour after dosing, von Frey assay, hind limb weight distribution and/or hind limb grip analysis was performed to measure the efficacy of the test compounds. An increase in paw withdrawal threshold (PWT) by a compound in comparison with vehicle is indicative of the test compound exhibiting therapeutic efficacy in the MIA model of osteoarthritis.

### Example 8—Compound with Low β-Arrestin Efficacy and Loss of In Vivo Efficacy

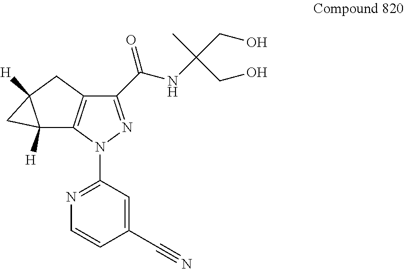 [SMILES: CC(CO)(CO)NC(=O)c1nn(-c2cc(C#N)ccn2)c2c1C[C@H]1C[C@@H]21]

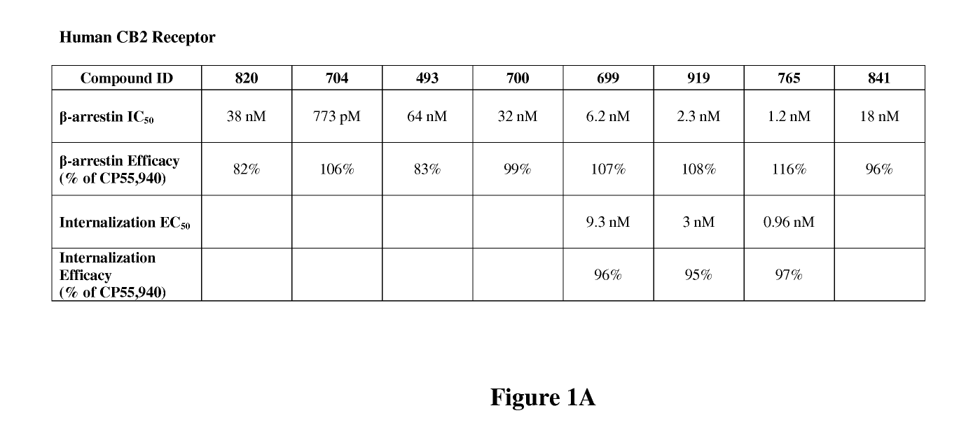

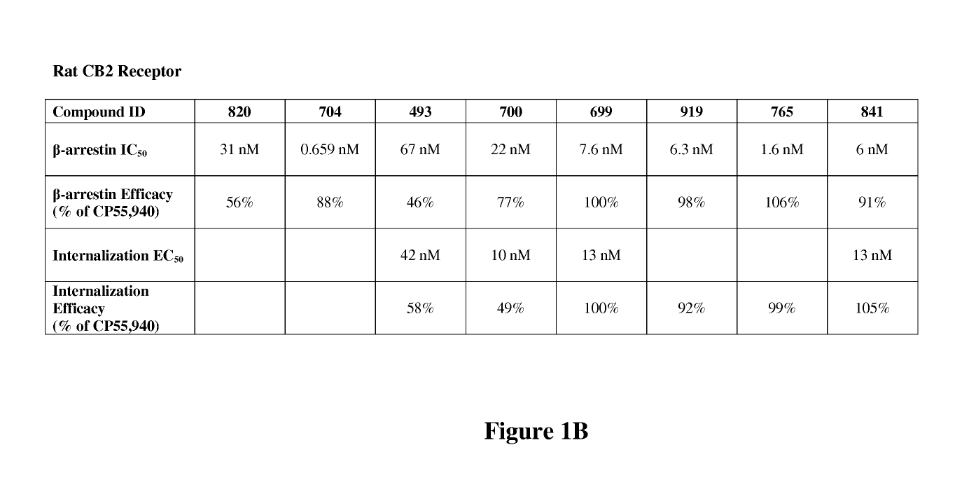

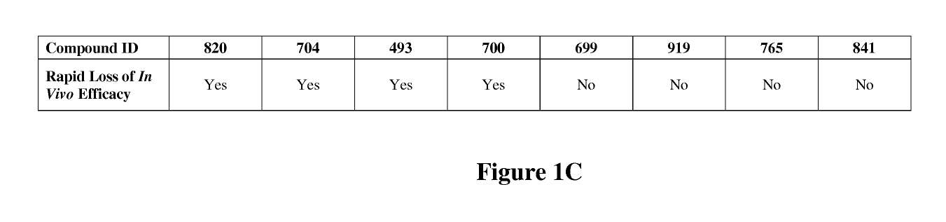

β-Arrestin assays were performed for mouse, rat, human CB2 and CB1 receptors following the administration of Compound 820. Compound 820 demonstrated 56% and 82% efficacy at the rat and human CB2 receptors, respectively, compared to CP55,940 (FIG. 1A).

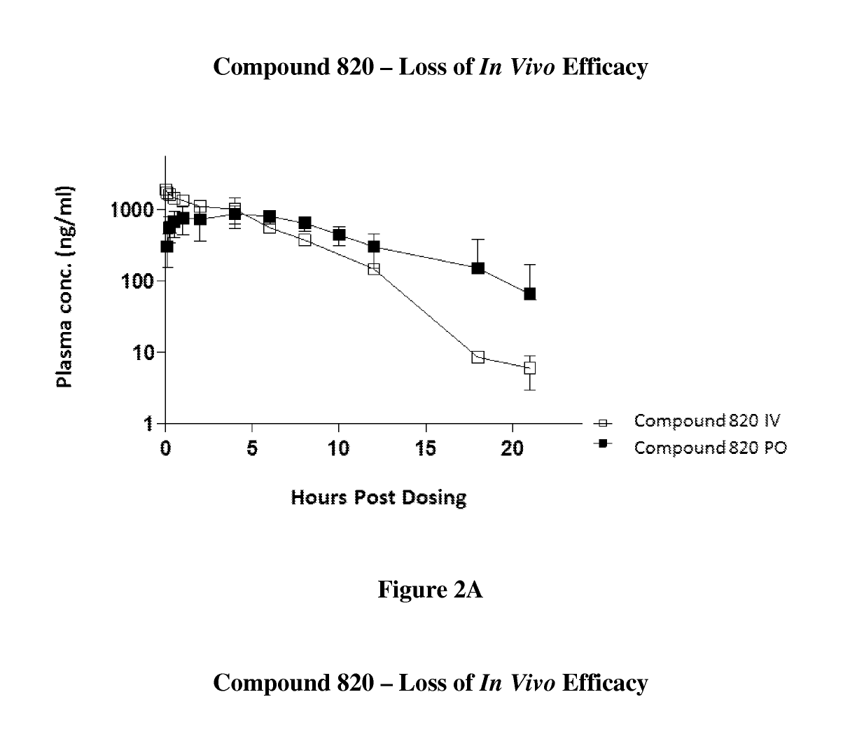

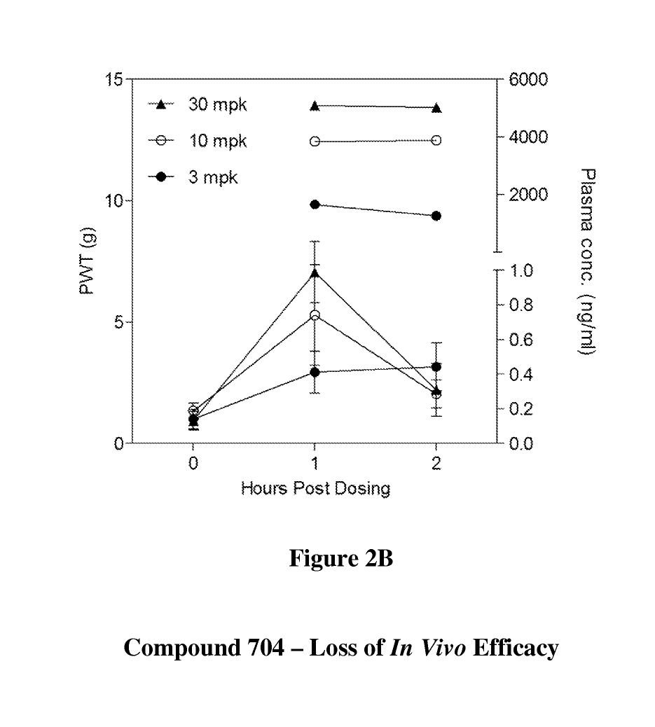

Compound 820 was also investigated in the osteoarthritis pain model. A rapid loss of in vivo efficacy was observed despite a high plasma concentration, with peak efficacy occurring one hour following dosing (FIGS. 2A-2B).

### Example 9—Compound with High β-Arrestin Efficacy and Loss of In Vivo Efficacy

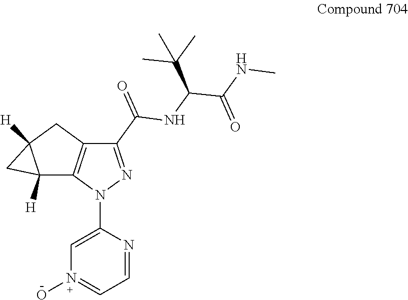

β-arrestin assays were performed for mouse, rat, human CB2 and CB1 receptors following the administration of Compound 704. Compound 704 demonstrated 88% and 106% efficacy at the rat and human CB₂ receptors, respectively, compared to CP55,940 (FIG. 1A).

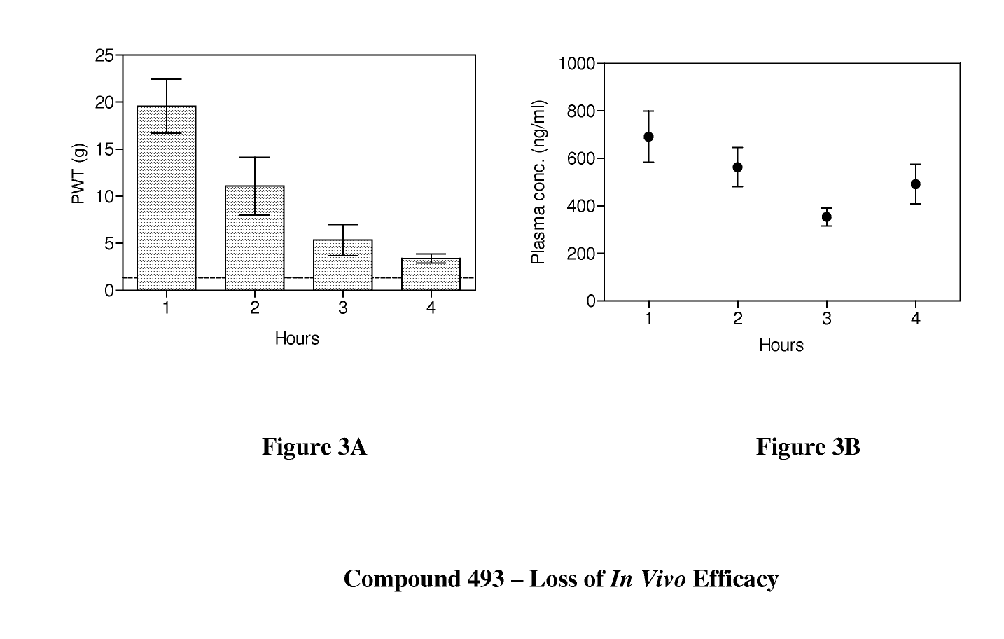

Compound 704 was also investigated in the osteoarthritis pain model. A rapid loss of in vivo efficacy was observed despite an increasing plasma concentration, with peak efficacy occurring one hour following dosing (FIGS. 3A-3B).

### Example 10—Compounds with Low Receptor Internalization and Loss of In Vivo Efficacy

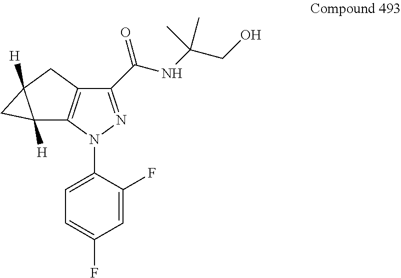 [SMILES: CC(C)(CO)NC(=O)c1nn(-c2ccc(F)cc2F)c2c1C[C@H]1C[C@@H]21]

β-arrestin assays were performed for mouse, rat, human CB2 and CB1 receptors following the administration of Compound 493, and CB2 receptor internalization assays were performed using conventional fluorescence microscopy for rat CB2 receptors following the administration of Compound 493. Compound 493 demonstrated a low receptor internalization efficacy for rat CB₂ receptors (58%) relative to CP55,940 (FIG. 1A).

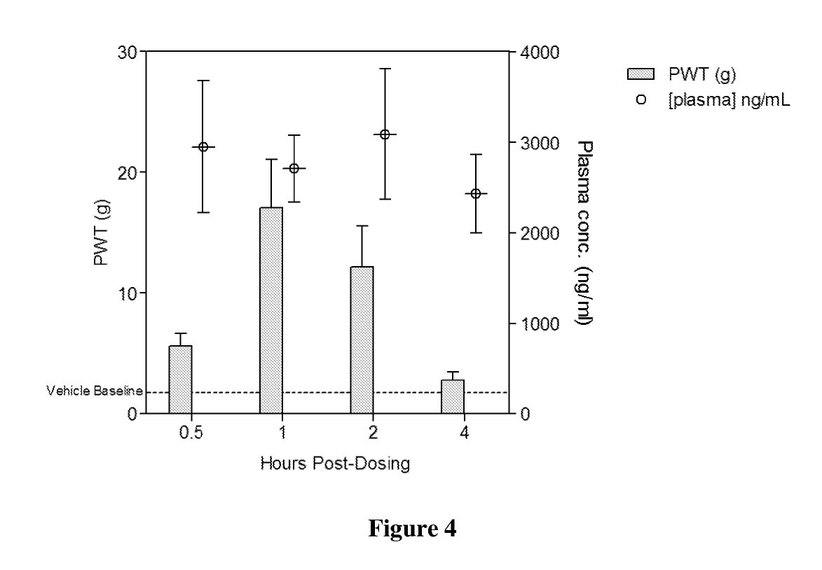

Compound 493 was also investigated in the osteoarthritis pain model. In vivo efficacy was completely lost between two and four hours following dosing, despite high plasma concentrations that did not decline significantly until four hours following dosing (FIG. 4).

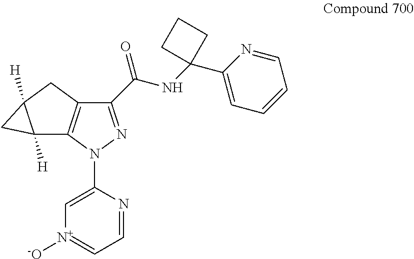

β-arrestin assays were performed for mouse, rat, human CB2 and CB1 receptors following the administration of Compound 700, and CB2 receptor internalization assays were performed using conventional fluorescence microscopy for rat CB2 receptors following the administration of Compound 700. Compound 700 demonstrated low receptor internalization efficacy for rat CB₂ receptors (49%) relative to CP55,940 (FIG. 1A).

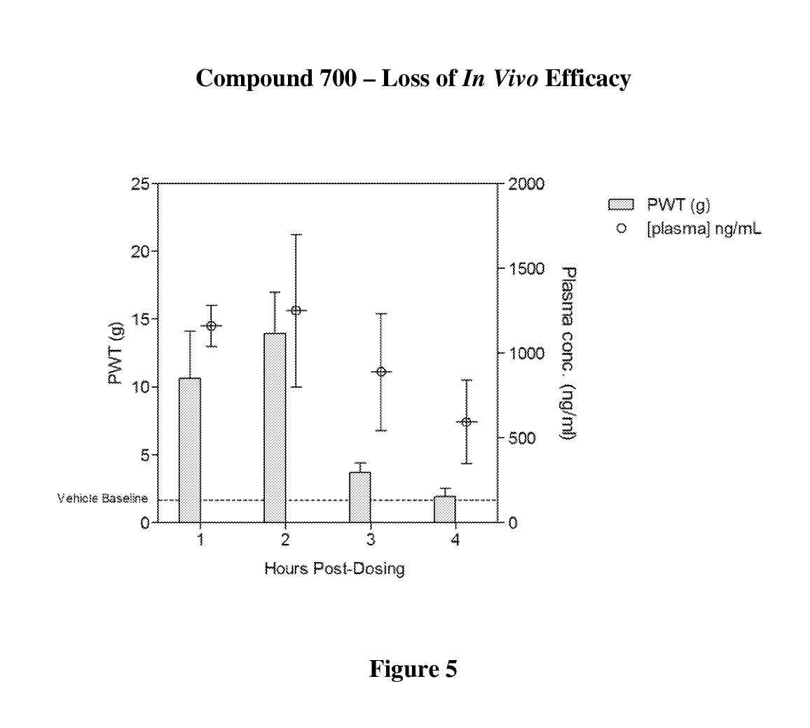

Compound 700 was also investigated in the osteoarthritis pain model. In vivo efficacy was lost around 3-4 hours following dosing, despite plasma concentrations that were no more than two times lower than at the two hour timepoint (FIG. 5).

### Example 11—Compounds with High Receptor Internalization and Sustained In Vivo Efficacy

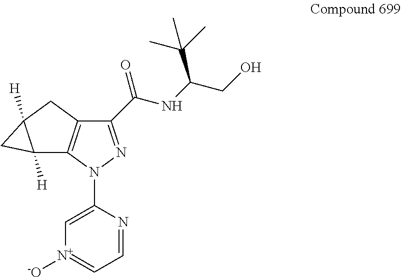

As described herein, Compound 699 has demonstrated sustained efficacy in models of osteoarthritis pain, paclitaxel-induced neuropathic pain, and painful peripheral diabetic neuropathy. Compound 699 also demonstrates >1,000-fold selectivity for the human CB2 receptor versus the human CB1 receptor.

β-arrestin assays were performed for mouse, rat, human CB2 and CB1 receptors following the administration of Compound 699, and CB2 receptor internalization assays were performed using conventional fluorescence microscopy for human and rat CB2 receptors following the administration of Compound 699. Compound 699 demonstrated a high receptor internalization efficacy for rat and human CB₂ receptors relative to CP55,940 (105% and 96%, respectively) (FIG. 1A).

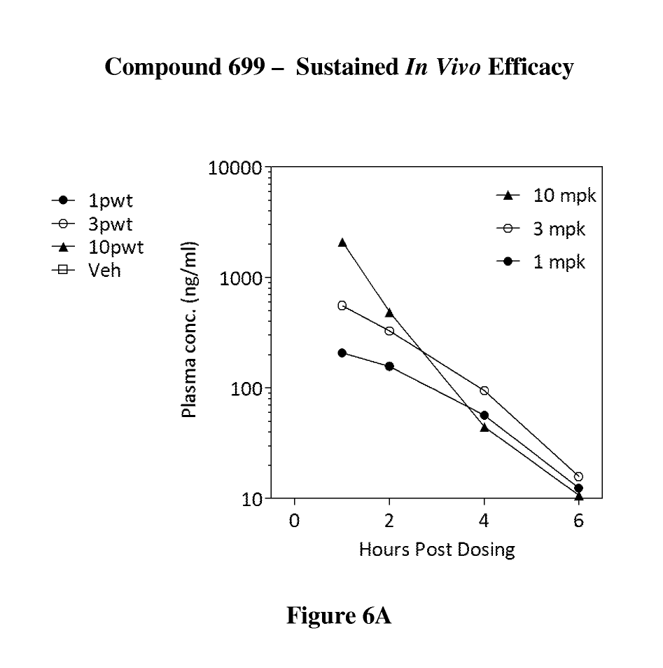

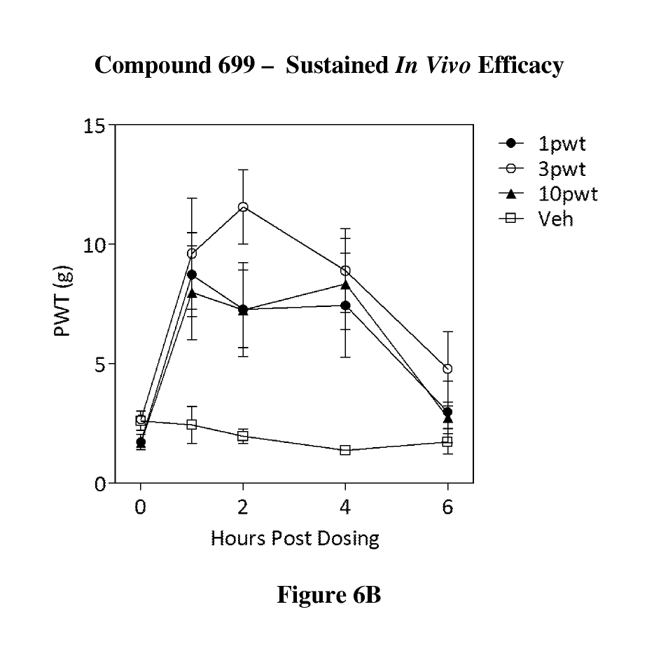

Compound 699 was also investigated in the osteoarthritis pain model. In vivo efficacy was maintained four hours following dosing, despite rapidly declining plasma concentrations (FIGS. 6A-B). In vivo efficacy was finally lost at the six hour timepoint, when plasma drug levels were <20 ng/mL (FIG. 6A).

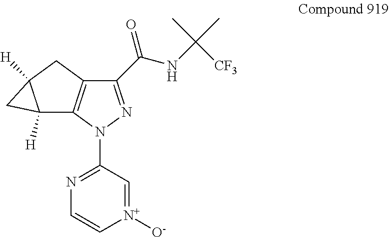 [SMILES: CC(C)(NC(=O)c1nn(-c2c[n+]([O-])ccn2)c2c1C[C@@H]1C[C@H]21)C(F)(F)F]

β-arrestin assays were performed for mouse, rat, human CB2 and CB1 receptors following the administration of Compound 919, and CB2 receptor internalization assays were performed using conventional fluorescence microscopy for human and rat CB2 receptors following the administration of Compound 919. Compound 919 demonstrated a high receptor internalization efficacy for rat and human CB₂ receptors relative to CP55,940 (92% and 95%, respectively) (FIG. 1A).

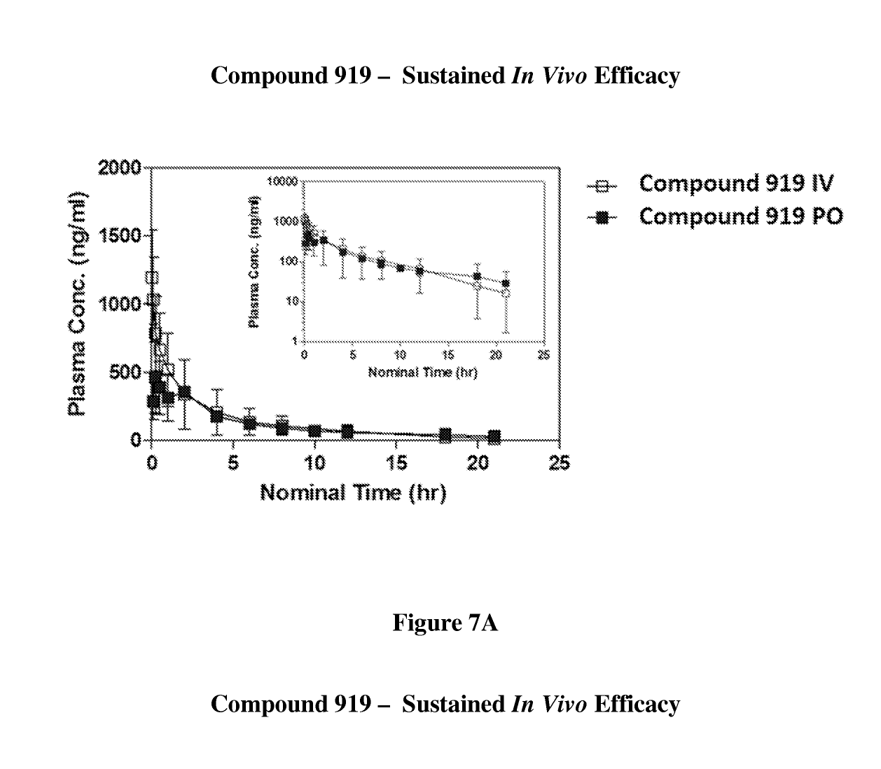

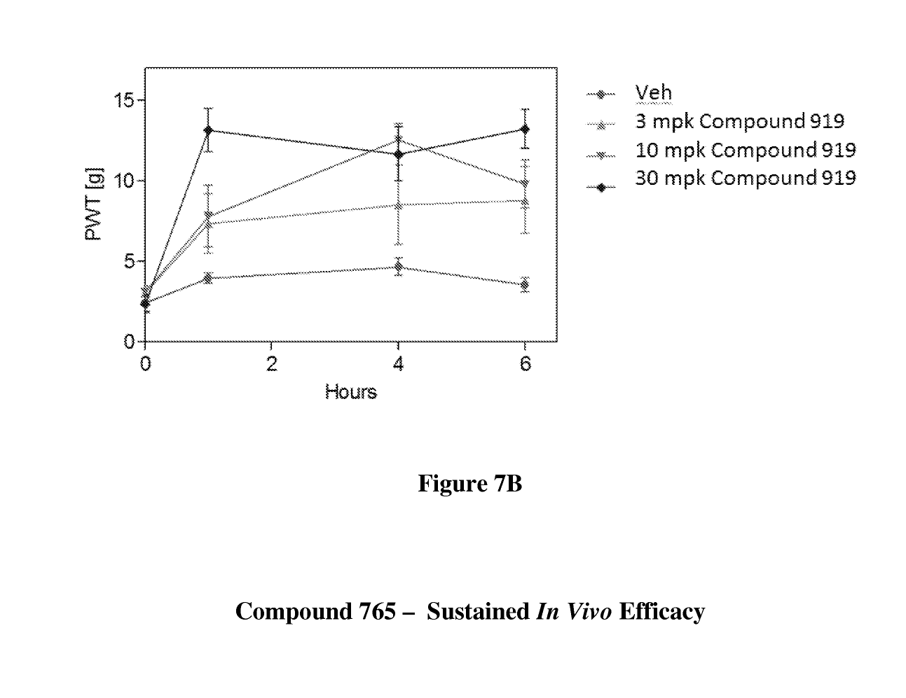

Compound 919 was also investigated in the osteoarthritis pain model. In vivo efficacy was maintained six hours post-dosing despite rapidly declining plasma concentrations (FIGS. 7A-B).

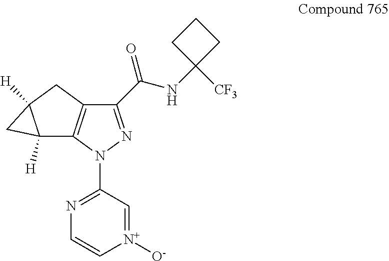 [SMILES: O=C(NC1(C(F)(F)F)CCC1)c1nn(-c2c[n+]([O-])ccn2)c2c1C[C@@H]1C[C@H]21]

β-arrestin assays were performed for mouse, rat, human CB2 and CB1 receptors following the administration of Compound 765, and CB2 receptor internalization assays were performed using conventional fluorescence microscopy for human and rat CB2 receptors following the administration of Compound 765. Compound 765 demonstrated a high receptor internalization efficacy for rat and human CB₂ receptors relative to CP55,940 (99% and 97%, respectively) (FIG. 1A).

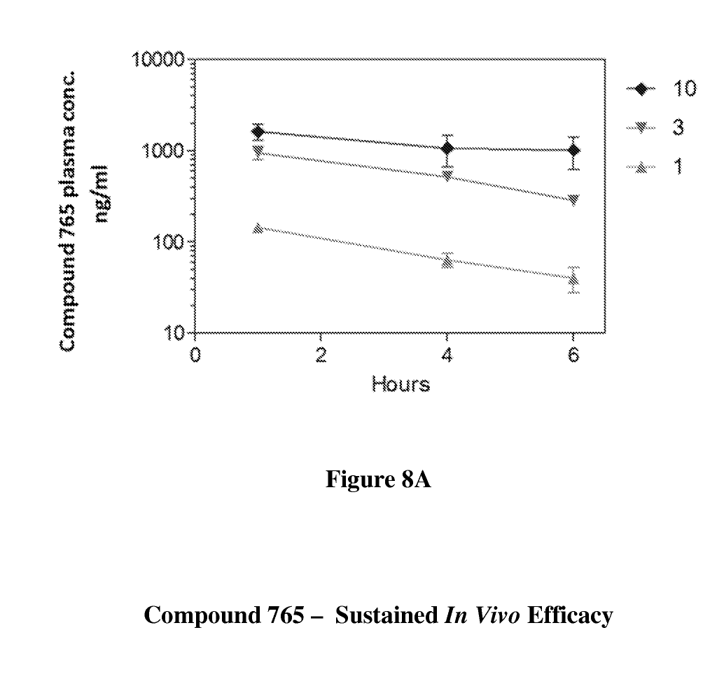

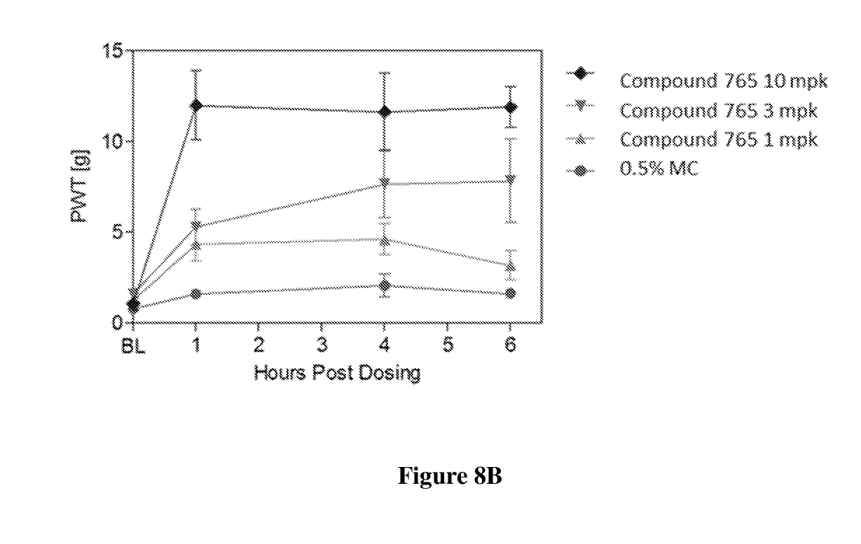

Compound 765 was also investigated in the osteoarthritis pain model. In vivo efficacy was maintained six hours following dosing (FIGS. 8A-B).

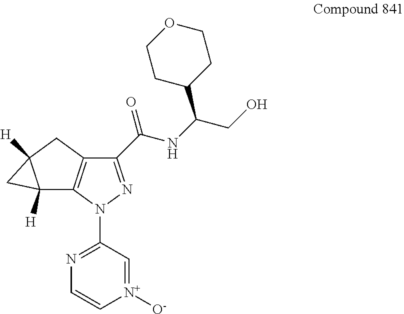 [SMILES: O=C(N[C@H](CO)C1CCOCC1)c1nn(-c2c[n+]([O-])ccn2)c2c1C[C@H]1C[C@@H]21]

β-arrestin assays were performed for mouse, rat, human CB2 and CB1 receptors following the administration of Compound 841, and CB2 receptor internalization assays were performed using conventional fluorescence microscopy for rat CB2 receptors following the administration of Compound 841. Compound 841 demonstrated a high receptor internalization efficacy for rat CB₂ receptors (105%) relative to CP55,940 (FIG. 1A).

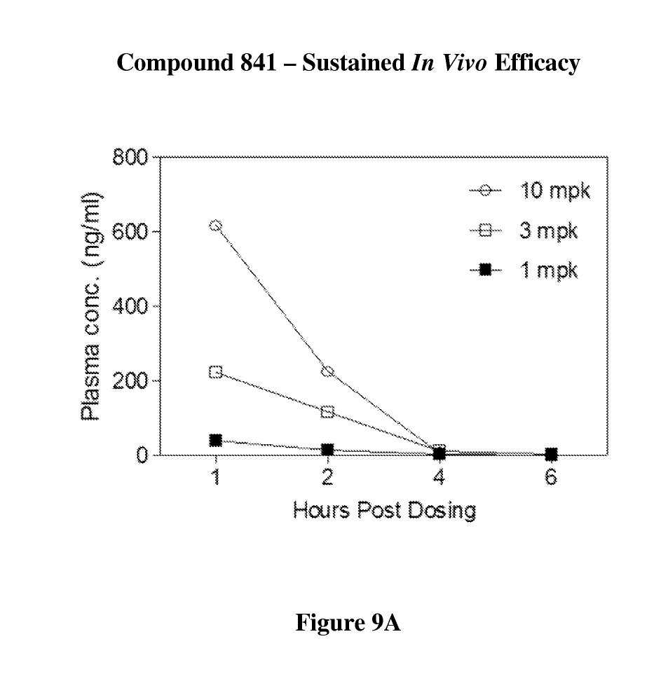

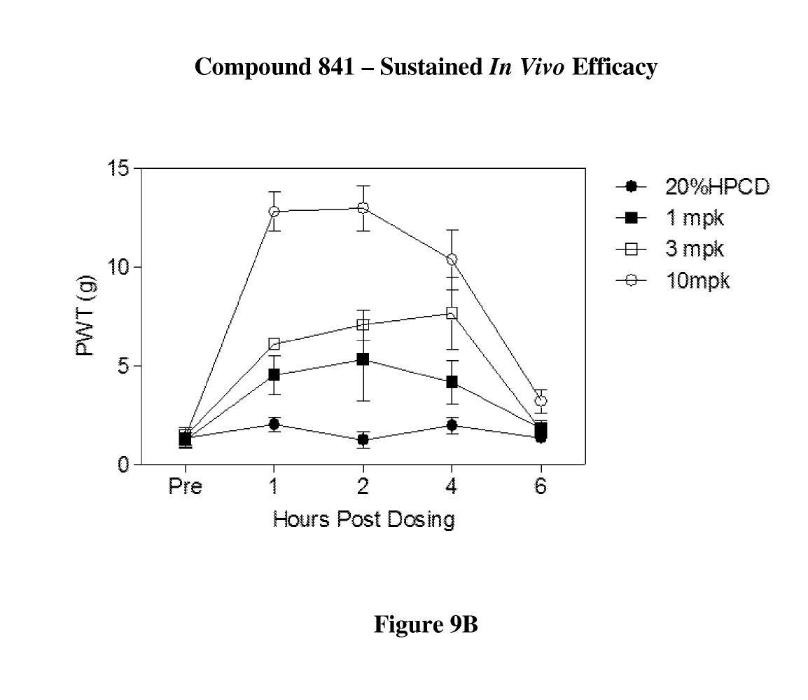

Compound 841 was also investigated in the osteoarthritis pain model. In vivo efficacy was maintained four hours post-dosing despite rapidly declining plasma concentrations (FIGS. 9A-B). Efficacy was finally lost at the six hour timepoint, when plasma drug levels were <3 ng/mL (FIG. 9A).

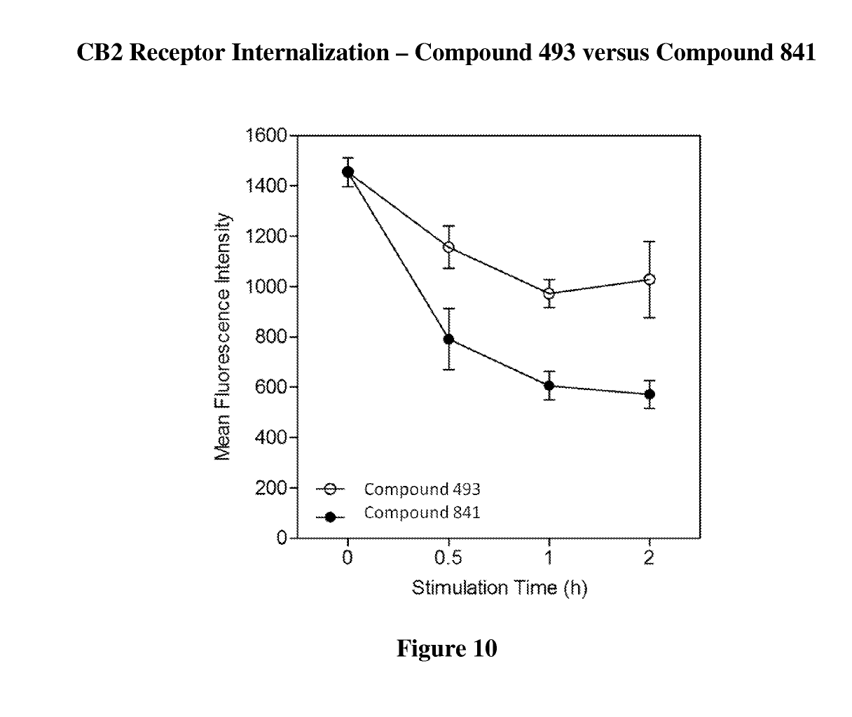

A direct comparison of receptor internalization for Compound 493 (which demonstrates low receptor internalization and rapid loss of in vivo efficacy) and Compound 841 (which demonstrates high receptor internalization and sustained in vivo efficacy) is provided in FIG. 10.

### Example 12—Clinical Candidates with In Vitro Assay Results Differing from Previous Reports

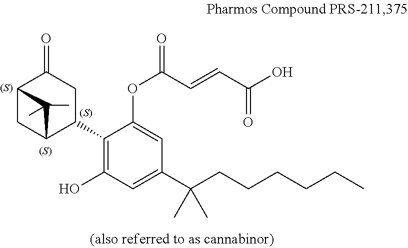

Cannabinor has been investigated in phase 1 clinical trials for post-operative (third molar extraction) pain, and in phase 2 clinical trials for capsaicin-induced pain. The trial for capsaicin-induced pain failed to meet the primary endpoint. Further, despite completing the phase 2 clinical trials in 2007, Pharmos has not advanced the compound in subsequent clinical trials.

Although there is only limited published data regarding cannabinor, one publication suggests that the compound is a full agonist at the human CB2 receptor and has 321-fold selectivity for the CB2 receptor over the CB1 receptor (17.4 nM vs. 5595 nM in GTP-γS binding assays). However, upon closer examination, the compound is only a partial agonist at the CB2 receptor (efficacy only appears to approach 100% because of a single data point with an extremely large error (Eur. Urol. 57:1093-1100 (2010), FIG. 2b), and the selectivity measured in radioligand binding assays appears to be approximately 10-fold (Eur. Urol. 57:1093-1100 (2010), FIG. 2a). Cannabinor is therefore unlikely to be a highly potent, selective CB2 receptor agonist.

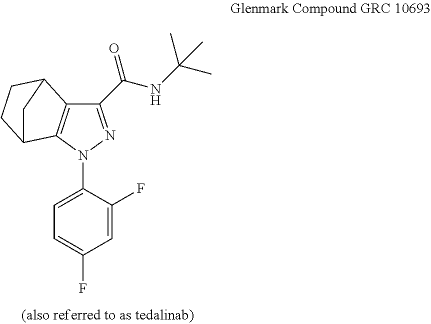 [SMILES: CC(C)(C)NC(=O)c1nn(-c2ccc(F)cc2F)c2c1C1CCC2C1]

GRC 10693 has been investigated in a phase 1 clinical trial for pain. Despite completing the phase 1 clinical trial in 2008, Glenmark has not advanced the compound in subsequent clinical trials.

Glenmark has reported that GRC 10693 is a highly potent molecule with a functional IC50 of 2.1 nM for the human CB2 receptor, and greater than 4700-fold selectivity compared to the CB1 receptor using cAMP assays (abstract submission to Society for Neuroscience, Oct. 14-18, 2006).

TABLE 1
β-Arrestin Assays for GRC 10693

Human CB2: 1.2 nM/100%
Human CB1: 384 nM/57%
Rat CB2: 2 nM/89%
Rat CB1: 290 nM/57%
Mouse CB2: 0.8 nM/96%
Mouse CB1: 750 nM/75%

β-arrestin assays were performed for mouse, rat, human CB2 and CB1 receptors following the administration of GRC 10693, and CB2 receptor internalization assays were performed using enzyme complementation for human and rat CB2 receptors following the administration of GRC 10693. GRC 10693 demonstrated high efficacy in the β-arrestin and receptor internalization assays relative to CP55,940 (FIG. 1B). However, GRC 10693 exhibited a lower selectivity for the human CB2 receptor (320-fold compared to the human CB1 receptor) than previous reports (Table 1).

### Example 13—Clinical Candidate with Low β-Arrestin Efficacy and Low Receptor Internalization

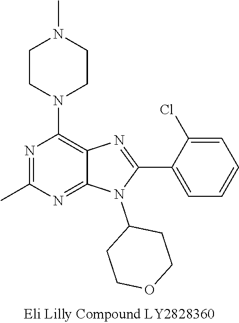 [SMILES: Cc1nc(N2CCN(C)CC2)c2nc(-c3ccccc3Cl)n(C3CCOCC3)c2n1]

TABLE 2
In Vitro Efficacy Reported by Lilly

GTPγS³⁵ Stimulation
hCB2: EC50 = 20.1 ± 3.2 nM
87 ± 2%
hCB1: EC50 > 100,000 nM
15 ± 7% at 100,000 nM
Displacement of [³H]CP55940
Human CB2: Ki = 40.3 ± 6.9 nM
Human CB1: Ki = 1300 ± 537 nM
Rat CB2; Ki = 36.7 ± 2.7 nM
Rat CB1: Ki = 560 ± 161 nM

LY2828360 has been investigated for osteoarthritis in a phase 2 clinical trial. Lilly has reported 87% in vitro efficacy for the human CB2 receptor using a GTP-γS binding assay, and 32-fold binding selectivity at the human CB2 receptor when measuring displacement of CP55,940 (Table 2) (J. Med. Chem. 56:5722-5733, 2013; Johnson et al., Neuroscience poster: A novel selective cB2 agonist, LY28283620 is efficacious in chronic pain models, 2012).

TABLE 3
β-Arrestin Assays for LY2828360

Human CB2: 3 nM/41%
Human CB1: 1.2 uM*
Rat CB2: 27 nM*
Rat CB1: 1.4 uM*
Mouse CB2: 56 nM*
Mouse CB1: 1 uM*
*indicates antagonist activity

β-arrestin assays were performed following the administration of LY2828360 to human, rat, and mouse CB1 and CB2 receptors, and CB2 receptor internalization assays were performed using enzyme complementation for human and rat CB2 receptors following the administration of LY2828360.

In the β-arrestin assay for the human CB2 receptor, LY2828360 demonstrated an EC50 of 3 nM an in vitro efficacy of only 41% compared to CP55,940 (Table 3; FIG. 1B). In the β-arrestin assays for the rat CB2 receptor, LY2828360 demonstrated an absence of agonist activity for the CB2 receptor compared to CP55,940 (FIG. 1B). In fact, LY2828360 essentially functioned as a CB2 receptor antagonist, demonstrating blockade of CP55,940-stimulated β-arrestin recruitment. The data observed for LY2828360 in the β-arrestin assays suggest an effect that is likely below the level required to avoid tachyphylaxis.

### Example 14—Clinical Candidate with High β-Arrestin Efficacy and Low Receptor Internalization

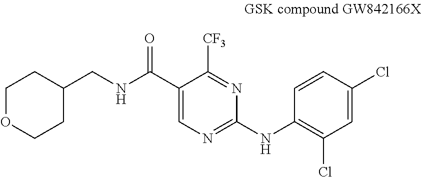 [SMILES: O=C(NCC1CCOCC1)c1cnc(Nc2ccc(Cl)cc2Cl)nc1C(F)(F)F]

GW842166X has been investigated in phase 2 clinical trials for osteoarthritis and dental pain. GSK has reported potencies of 63 nM and 91 nM for GW842166X at the human and rat CB2 receptors, respectively, using cAMP assays (J. Med. Chem. 50:2597-2600, 2007).

TABLE 4
β-Arrestin Assays for GW842166X

Human CB2: 840 nM/99%
Human CB1: >10 uM
Rat CB2: 427 nM/97%
Rat CB1: >10 uM
Mouse CB2: 235 nM/108%
Mouse CB1: >10 uM

β-arrestin assays were performed for mouse, rat, human CB2 and CB1 receptors following the administration of GW842166X, and CB2 receptor internalization assays were performed using enzyme complementation for human and rat CB2 receptors following the administration of GW842166X. GW842166X demonstrated a surprisingly low potency of 840 nM, but with 99% efficacy at the human CB2 receptor using the β-arrestin assay (Table 4). However, GW842166X drove weak receptor internalization, with a total granule intensity of only 53% compared to CP55,940 for the human CB2 receptor (FIG. 1B).

GW842166X was also investigated in the osteoarthritis pain model. Although GSK has reported an absence of CB1 activity with concentrations of GW842166X less than 30 μM, the limited efficacy for GW842166X observed in the pain model was blocked by CB1 antagonists—suggesting that the effects of GW842166X are CB1 receptor-dependent.

What is claimed is:

1. A method comprising:
measuring internalization of CB2 receptors in a cell following contact with a compound; and
formulating the compound into a pharmaceutical composition if internalization of the CB2 receptors is increased to a predefined level following contact with the compound.

2. A method comprising:
selecting a compound previously identified as increasing internalization of CB2 receptors to a predefined level in a cell; and
formulating the compound into a pharmaceutical composition.

3. A method comprising:
selecting a compound previously identified as:
increasing internalization of CB2 receptors in a cell; and
demonstrating in vivo efficacy in a mammal; and
formulating the compound into a pharmaceutical composition.

4. A method comprising:
measuring internalization of CB2 receptors in a cell following contact with a compound; and
producing, isolating, or synthesizing the compound if the compound increases internalization of the CB2 receptors to a predefined level in the cell.

5. A method comprising:
measuring internalization of CB2 receptors in a cell following contact with a compound;
administering a compound that increases internalization of the CB2 receptors to a predefined level to a mammal; and
formulating the compound into a pharmaceutical composition if the compound demonstrates in vivo efficacy in the mammal.

6. A method comprising:
selecting a compound identified as increasing internalization of CB2 receptors to a predefined level in a cell; and
administering the compound to an individual in need thereof.

7. The method of any one of claim 1-4 or 6, further comprising:
administering a compound that increases internalization of CB2 receptors to the predefined level to a mammal; and
measuring efficacy of the compound in the mammal.

8. The method of any one of claims 1-7, further comprising:
measuring selectivity of the compound for the CB2 receptor relative to the CB1 receptor.

9. The method of any one of claims 1-7, further comprising:
measuring selectivity of the compound for the human CB2 receptor relative to the human CB1 receptor.

10. The method of any one of claims 1-7, wherein the compound has previously been identified as exhibiting at least 500-fold, at least 750-fold, at least 1000-fold, at least 2000-fold, at least 3000-fold, at least 4000-fold, at least 5000-fold, at least 6000-fold, at least 7000-fold, at least 8000-fold, at least 9000-fold, or at least 10,000-fold selectivity for the human CB2 receptor relative to the human CB1 receptor.

11. The method of any one of claim 1-3, 5, or 7-10, wherein the compound in the pharmaceutical composition is in an amount sufficient for the treatment or prevention of a CB2 receptor-mediated disorder.

12. The method of claim 6, wherein the compound is in an amount sufficient for the treatment or prevention of a CB2 receptor-mediated disorder.

13. The method of any one of claims 1-12, wherein the ability of a compound to increase internalization of the CB2 receptors to the predefined level is indicative of the compound being useful for the treatment of a CB2 receptor-mediated disorder.

14. The method of any one of claims 1-12, wherein the compound is suitable for the treatment of a CB2 receptor-mediated disorder.

15. The method of claim 14, wherein the CB2 receptor-mediated disorder is pain, fibrosis, or a condition related thereto.

16. The method of claim 14, wherein the CB2 receptor-mediated disorder is selected from: pain associated with osteoarthritis, neuropathic pain, acute post-operative pain, liver fibrosis, primary biliary cirrhosis, nonalcoholic steatohepatitis, renal fibrosis, endometriosis, and interstitial cystitis.

17. The method of any one of claim 3, 5, or 7-16, wherein the mammal is a non-human mammal.

18. The method of any one of claim 3, 5, or 7-16, wherein the mammal is a human.

19. The method of any one of claims 1-18, wherein the CB2 receptors are human CB2 receptors.

20. The method of any one of claims 1-18, wherein the CB2 receptors are recombinant.

21. The method of claim 20, wherein the CB2 receptors are encoded by a nucleotide sequence comprising at least 75%, at least 80%, at least 85%, at least 90%, at least 95%, at least 96%, at least 97%, at least 98%, at least 99%, or 100% identity to NCBI Reference Sequence NM_001841.

22. The method of any one of claims 1-21, wherein the CB2 receptors comprise a sequence with at least 75%, at least 80%, at least 85%, at least 90%, at least 95%, at least 96%, at least 97%, at least 98%, at least 99%, or 100% identity to NCBI Reference Sequence NP_001832.1.

23. The method of any one of claims 1-22, wherein the compound is non-naturally occurring.

24. The method of any one of claims 1-23, wherein the compound is orally active.

25. The method of any one of claims 1-24, wherein the cell is a liver cell, a kidney cell, or a lung cell.

26. The method of any one of claims 1-25, wherein internalization is measured, or has been measured, relative to the level of internalization that would occur if the cell were contacted with a full CB2 receptor internalization agonist.

27. The method of any one of claims 1-25, wherein internalization is measured, or has been measured, relative to the level of internalization that would occur if the cell were contacted with CP55,940.

28. The method of any one of claims 1-27, wherein the compound increases internalization of the CB2 receptors to at least 75%, at least 80%, at least 85%, at least 90%, at least 95%, at least 96%, at least 97%, at least 98%, or at least about 99% the level of internalization that would occur if the cell were contacted with CP55,940.

29. The method of any one of claim 1, 4, 5, or 7-28, wherein internalization is measured using a method selected from: flow cytometry, fluorescence microscopy, and enzyme complementation.

30. The method of any one of claims 7-29, wherein efficacy of the compound in the mammal is measured at least about 2 hours, at least about 3 hours, at least about 4 hours, at least about 5 hours, or at least about 6 hours following administration to the mammal.

31. The method of any one of claims 7-29, wherein the mammal is an animal model for pain, fibrosis, or a condition related thereto.

32. A pharmaceutical composition prepared according to any one of claim 1-5 or 7-31.

33. A compound identified as increasing internalization to a predefined level according to any one of claim 1, 4, 5, or 7-31.

34. A pharmaceutical composition comprising a compound according to claim 33.

35. A pharmaceutical composition comprising a compound according to claim 33 and a pharmaceutical excipient.

36. The pharmaceutical composition according to any one of claim 32, 34, or 35, wherein the pharmaceutical composition is suitable for oral, rectal, nasal, topical, buccal, sub-lingual, or vaginal, or in a form suitable for administration by inhalation, insufflation, or by a transdermal patch.

37. The pharmaceutical composition according to any one of claim 32, 34, or 35, wherein the pharmaceutical composition is suitable for oral administration.

38. A process for preparing a pharmaceutical composition, comprising admixing a pharmaceutically acceptable carrier with a compound selected as increasing internalization of CB2 receptors in a cell to at least 75%, at least 80%, at least 85%, at least 90%, at least 95%, at least 96%, at least 97%, at least 98%, or at least about 99% the level of internalization that would occur if the cell were contacted with CP55,940.

39. A process for preparing a pharmaceutical composition, comprising admixing a pharmaceutically acceptable carrier with a compound selected as increasing internalization of CB2 receptors in a cell to at least 75%, at least 80%, at least 85%, at least 90%, at least 95%, or at least about 99% the level of internalization that would occur if the cell were contacted with CP55,940, wherein the compound has also been selected as exhibiting at least 50-fold, at least 100-fold, at least 500-fold, at least 750-fold, at least 1000-fold, at least 5000-fold, or at least 10,000-fold selectivity for the human CB2 receptor relative to the human CB1 receptor.

40. The process of claim 38 or 39, wherein the compound is in an amount sufficient for the treatment or prevention of a CB2 receptor-mediated disorder.

41. The process of claim 40, wherein the CB2 receptor-mediated disorder is pain, fibrosis, or a condition related thereto.

42. Use of a compound selected as increasing internalization of CB2 receptors in a cell to at least 75%, at least 80%, at least 85%, at least 90%, at least 95%, or at least about 99% the level of internalization of CB2 receptors following contact with CP55,940 in the manufacture of a medicament for treating or preventing a CB2 receptor-mediated disorder in an individual.

43. The use according to claim 42, wherein the CB2 receptor-mediated disorder is pain, fibrosis, or a condition related thereto.

44. Use of a compound selected as increasing internalization of CB2 receptors in a cell to at least 75%, at least 80%, at least 85%, at least 90%, at least 95%, or at least about 99% the level of internalization that would occur following contact with CP55,940, wherein the compound has been also selected as exhibiting at least 50-fold, at least 100-fold, at least 500-fold, at least 750-fold, at least 1000-fold, at least 5000-fold, or at least 10,000-fold selectivity for the human CB2 receptor relative to the human CB1 receptor, in the manufacture of a medicament for treating or preventing a CB2 receptor-mediated disorder in an individual.

45. The use according to claim 44, wherein the CB2 receptor-mediated disorder is pain, fibrosis, or a condition related thereto.

46. A compound for use in the treatment of a CB2 receptor-mediated disorder in an individual, wherein the compound has been selected as increasing internalization of CB2 receptors to at least 75%, at least 80%, at least 85%, at least 90%, at least 95%, or at least about 99% the level of internalization that would occur following contact with CP55,940, with a pharmaceutically acceptable carrier.

47. A compound for use in the treatment of a CB2 receptor-mediated disorder in an individual, wherein the compound has been selected as increasing internalization of CB2 receptors to at least 75%, at least 80%, at least 85%, at least 90%, at least 95%, or at least about 99% the level of internalization that would occur following contact with CP55,940, and wherein the compound has also been selected as exhibiting at least 50-fold, at least 100-fold, at least 500-fold, at least 1000-fold, at least 5000-fold, or at least 10,000-fold selectivity for the human CB2 receptor relative to the human CB1 receptor, with a pharmaceutically acceptable carrier.

48. The compound for use according to claim 46 or 47, wherein the CB2 receptor-mediated disorder is pain, fibrosis, or a condition related thereto.
